(19) United States
(12) Patent Application Publication
(10) Pub. No.: US 2020/0323830 A1
(43) Pub. Date: Oct. 15, 2020

(54) TREATMENT OF VIRAL HEMORRHAGIC FEVERS WITH ETORICOXIB

(71) Applicant: Tremeau Pharmaceuticals, Inc., Concord, MA (US)

(72) Inventors: Bradford C. SIPPY, Acton, MA (US); Travis E. HELM, Acton, MA (US)

(21) Appl. No.: 16/843,808

(22) Filed: Apr. 8, 2020

Related U.S. Application Data
(60) Provisional application No. 62/831,407, filed on Apr. 9, 2019.

(51) Int. Cl. A61K 31/444; A61K 9/20; A61K 9/48; A61K 9/08; A61K 31/167

(52) U.S. Cl. CPC A61K 31/444; A61K 9/20; A61K 31/167; A61K 9/08; A61K 9/48

(57) ABSTRACT

The subject matter disclosed herein relates to methods of treating viral hemorrhagic fever in a subject in need thereof by administering a pharmaceutically acceptable dose of etoricoxib. In some embodiments, the etoricoxib is administered in combination with other suitable pharmaceutical compositions.

This application claims the benefit of and priority to U.S. Provisional Patent Application No. 62/831,407 filed on Apr. 9, 2019, the content of which is hereby incorporated by reference in its entirety.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Viral Hemorrhagic Fevers

Viral hemorrhagic fevers (VHFs) fevers are infectious diseases that can cause severe, life-threatening illness. Symptoms arising from VHF infections include high fever and muscle, bone or joint aches, which may be accompanied by hemorrhage and/or bleeding. There are currently no approved, curative treatments for subjects infected with VHFs. Acetaminophen (paracetamol) is recommended for the treatment of symptoms of fever and pain associated with VHFs.

VHFs are caused by RNA viruses derived from four viral families linked by a clinical syndrome. These four families include the Arenaviridae, Bunyaviridae, Filoviridae, and Flaviviridae. The Arenaviridae include etiologic agents of Argentine, Bolivian and Venezuelan hemorrhagic fevers and Lassa fever. The Bunyaviridae include members of the Hantavirus genus, the Congo-Crimean Hemorrhagic fever virus from the Nairovirus genus, and the Rift Valley fever virus from the Phlebovirus genus. The Filoviridae include Ebola and Marburg viruses. The Flaviviridae include Dengue and Yellow fever viruses. These viruses are spread to humans in a variety of ways including, but not limited to, direct contact (i.e., droplet transmission from an infected human), indirect contact (i.e., animal to human and insect vector to human) or transmission via entry of a respiratory portal. A summary further describing the four key VHFs is provided below.

Arenaviridae

Argentine Hemorrhagic Fever (AHF), caused by the Junin virus, was discovered in 1955, following reports of AHF symptoms found in corn harvesters. Today, nearly 300 to 600 cases per year are reported in regions of the Argentine pampas. The Bolivian, Brazilian and Venezuelan Hemorrhagic Fevers are caused by related Machupo, Guanarito and Sabia viruses, with the Lassa virus being a common cause for disease in West Africa. These viruses are transmitted from their rodent reservoirs to humans by the inhalation of dust particles contaminated with rodent excreta.

Bunyaviridae

Congo-Crimean Hemorrhagic Fever (CCHF) is primarily a tick-borne disease found in Crimea and in parts of Africa, Europe and Asia. However, CCHF can also be spread by contact with infected animals and in healthcare facilities treating CCHF infected patients. Rift Valley Fever (RVF) is a mosquito-borne disease in Africa. The hantaviruses are rodent-borne viruses with a wide geographic distribution. Hantaan, and closely related viruses, cause hemorrhagic fever with renal syndrome (HFRS, also known as Korean Hemorrhagic Fever or Epidemic Hemorrhagic Fever) and is reportedly the most common disease due to the hantaviruses. Prior to World War II, Hantaan was described as being found in Manchuria along the Amur River, infected among United Nations troops during the Korean conflict, and subsequently in Japan, China and in the Russian Far East. Severe disease also occurs in some Balkan states, including Bosnia, Serbia and Greece. Nephropathia epidemica is a milder disease found in Scandinavia and in other parts of Europe, and is caused by strains carried by bank voles. In addition, newly described hantaviruses cause Hantavirus Pulmonary Syndrome (HPS) in the Americas. The hantaviruses are also transmitted to humans by the inhalation of dust particles contaminated with rodent excreta.

Filoviridae

Ebola hemorrhagic fever was first recognized in 1976, in the western equatorial province of Sudan and the nearby region of Zaire. A second outbreak occurred in Sudan in 1979, and larger outbreak in 1995, comprising of at least 316 cases, which developed in Kikwit, Zaire from a single index case. Subsequent epidemics have also occurred in Gabon, the Ivory Coast, Uganda and the Republic of Congo. There are five species of Ebola: Zaire, Sudan, Ivory Coast, Reston and Bundibugyo. These African strains can cause severe disease and even death, with case fatality rates that vary by viral species (i.e., Bundibugyo ˜35%, Sudan 40%-50%, Zaire 80%-90%). At this time, it is not known why this disease appears infrequently. A related virus, the Ebola Reston, was isolated from monkeys in 1989, and these monkeys subsequently developed hemorrhagic fever. While subclinical infections occurred among exposed animal handlers, Ebola Reston has not been identified as a human pathogen despite being shown to cause disease in pigs and non-human primates. Marburg epidemics have occurred on eight separate occasions: six times in Africa and twice in Europe. The first recognized outbreak occurred in Marburg, Germany and in Yugoslavia among people exposed to African green monkeys; which resulted in 31 contracted cases and seven deaths. Overall, case fatality rates of the outbreaks in Marburg have varied from 21% to nearly 90%. While there is only one species of the Marburg virus, there exists several strains. Filoviruses can be spread from human to human by direct contact with infected blood, secretions, organs, or semen. Ebola Reston apparently spread from monkey to monkey, as well as from monkey to human by the respiratory route. While the natural reservoirs of the filoviruses are unknown, recent evidence strongly implicates bats as either the reservoir or as the intermediate host.

Flaviviridae

Yellow and Dengue fever are two mosquito-borne hemorrhagic fevers of the Flaviviridae family of viruses that have great importance in the history of military campaigns and military medicine, as well as in port cities engaging in commerce with the tropics, such as, for example, in New Orleans. Tick-borne hemorrhagic flaviviruses include the agents of Kyanasur Forest disease in India, and Omsk Hemorrhagic Fever in Siberia. The Zika virus is a non-hemorrhaging member of the Flaviviridae family.

Dengue fever is a mosquito-borne tropical disease caused by the Dengue virus, and is a member of the Flaviviridae family of viruses. Symptoms usually last up to 14 days and may include a sudden high-grade fever, headache, vomiting, rash, and extreme pain and stiffness of the joints. Though generally yielding a lower mortality rate than some other VHFs, the extreme pain and joint inflammation associated with Dengue fever has earned it the pseudonym “Breakbone Fever.” Symptoms in severe cases may include internal and external bleeding (i.e., Dengue hemorrhagic fever). An estimated 96 million clinical cases of Dengue fever occur annually worldwide and it is a leading cause of childhood death in Asia and Latin America. As Dengue fever has been a highly prevalent and well-known disease to modern medicine for many decades, significant detail regarding the time course of the disease, symptom manifestation, and relationship between viral load and fever level to the severity of the disease has been elucidated. Following an estimated 4-10 day incubation period after infection, the time course of Dengue fever follows three distinct phases: the febrile phase, the critical phase, and the recovery phase.

During the febrile phase, patients typically will rapidly develop a high-grade fever. This acute febrile phase usually lasts between about 2-7 days and is often accompanied by facial flushing, skin erythema, generalized body ache, myalgia, severe arthralgia and headache. Some patients may also experience a sore throat, injected pharynx and conjunctival injection. Anorexia, nausea and vomiting are also common. Because of this, it can be difficult to clinically distinguish Dengue from other hemorrhagic fevers in the early febrile phase. However, a positive tourniquet test, indicating vascular fragility, in this phase increases the probability of differential Dengue diagnosis. In addition, these clinical features are indistinguishable between severe and non-severe Dengue cases. Mild hemorrhagic manifestations like petechiae and mucosal membrane bleeding (e.g., nose and gums) may also be seen. Even more, extremely heavy vaginal bleeding (particularly in women of childbearing age) and gastrointestinal bleeding may occur during this phase, but is not common, and the liver may often become enlarged and tender after a few days of fever. In addition to primary manifestations of Dengue, secondary disease manifestations related to high fever (e.g., febrile seizures, especially in children) can occur at this phase.

The critical phase usually commences on day 3-7 of the illness after the internal temperature of the patient drops to 37.5-38 degrees C. or less and remains below this level. At that time, an increase in capillary permeability in parallel with increasing hematocrit levels may occur. If it occurs, the period of clinically significant plasma leakage initiates at this phase and will usually last between about 24-48 hours. Though uncommon, shock may occur when a critical volume of plasma is lost through leakage. With prolonged shock, the consequent organ hypoperfusion results in progressive organ impairment, metabolic acidosis and disseminated intravascular coagulation. This in turn leads to severe hemorrhage causing the hematocrit to decrease in severe shock. In addition, severe organ impairment, such as, for example, severe hepatitis, encephalitis or myocarditis and/or severe bleeding may develop without obvious plasma leakage or shock.

Patients with severe Dengue fever may have coagulation abnormalities, but these are usually not sufficient enough to cause major bleeding. When major bleeding does occur, it is almost always associated with profound shock because this, in combination with thrombocytopenia, hypoxia and acidosis, can lead to multiple organ failure and advanced disseminated intravascular coagulation. Massive bleeding may occur without prolonged shock in instances when acetylsalicylic acid (aspirin), ibuprofen or corticosteroids have been taken.

The recovery phase is characterized by a gradual reabsorption of extravascular compartment fluid over 2-3 days after the critical phase ends. At that time, the general well-being of the patient improves, appetite returns, gastrointestinal symptoms abate, hemodynamic status stabilizes, and diuresis ensues. Some patients may have a rash of what is known as the “isles of white in the sea of red,” while others may experience generalized pruritus. Bradycardia and electrocardiographic changes are also common during this stage.

It is thought that the risk of progression to more severe forms of Dengue fever is correlated with fever severity (i.e., fevers greater than 38 degrees C. associated with risk of severe Dengue) and viral load (i.e., viral loads higher during days 1-2 for severe versus normal Dengue) during the Febrile phase. In addition, it is thought that the risk of some secondary disease manifestations (e.g., febrile seizures) is correlated to fever severity during the Febrile Phase.

The Zika virus (ZIKV) is a member of the Flaviviridae family of viruses and is related to Dengue fever and Yellow fever. Its name comes from the Zika Forest of Uganda, where the virus was first isolated in 1947. The infection, also known as Zika fever or Zika virus disease, often causes no or only mild symptoms similar to a very mild form of Dengue fever. While Zika, in and of itself, does not manifest into a hemorrhagic fever because it is endemic in similar areas to Dengue and its early clinical onset is similar, treatment constraints applied to Dengue, such as avoidance of NSAIDs, are also applied to Zika.

### BRIEF DESCRIPTION OF THE FIGURES

FIG. 1 shows a flow diagram for the treatment of viral hemorrhagic fever with etoricoxib.

FIG. 2 shows an exemplary schematic of the study.

FIG. 3 shows the schedule of subject assessments.

FIG. 4 shows minimization of risk strategy.

FIG. 5 shows World Health Organization Toxicity Criteria.

### SUMMARY OF THE INVENTION

In certain aspects, the invention provides a method for treating one or more symptoms in a subject having viral hemorrhagic fever (VHF), the method comprising administering to the subject a pharmaceutical composition comprising a therapeutically effective amount of etoricoxib or a pharmaceutically acceptable salt thereof.

In some embodiments, the pharmaceutical composition further comprises a pharmaceutically acceptable carrier. In some embodiments, the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

In some embodiments, the VHF is caused by one or more RNA viruses derived from a family of viruses selected from Arenaviridae, Bunyaviridae, Filoviridae, Flaviviridae, and any combinations thereof. In some embodiments, the VHF is caused by one or more RNA viruses from the Flaviviridae family of viruses. In some embodiments, the VHF is caused by a Dengue Virus.

In some embodiments, the therapeutically effective amount of the etoricoxib causes a reduction in viral load, viral titer, or viral shedding. In some embodiments, the pharmaceutical composition is administered at a duration and a dosage such that the subject experiences a reduction in the one or more symptoms. In some embodiments, the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

In some embodiments, the pharmaceutical composition is provided in a quantity sufficient for a single course of therapy. In some embodiments, the pharmaceutical composition is provided in a single course of therapy package.

In some embodiments, the pharmaceutical composition is administered at a dosing schedule matched to the time course of the VHF. In some embodiments, the pharmaceutical composition comprises a dose of etoricoxib sufficient to achieve maximum anti-pyretic effect during the febrile phase of the infection. In some embodiments, the method further comprises administering to the subject a lower maintenance dose during the critical phase and/or recovery phase of the infection compared to the dose administered during the febrile phase of the infection. In some embodiments, the pharmaceutical composition comprising etoricoxib is administered at a dose that does not affect platelet aggregation or bleeding time.

In some embodiments, the pharmaceutical composition is administered at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

In some embodiments, the pharmaceutical composition is administered as an initial loading dose of about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day. In some embodiments, the initial loading dose is administered during the febrile phase of the infection.

In some embodiments, the method further comprises administering a maintenance dose of etoricoxib at about 30 mg/day, about 40 mg/day, about 50 mg/day about 60 mg/day, about 70 mg/day, about 80 mg/day, about 90 mg/day, about 100 mg/day, about 110 mg/day, or about 120 mg/day. In some embodiments, the maintenance dose is administered for up to 3 days, up to 5 days, up to 7 days, up to 10 days, or up to 14 days. In some embodiments, the maintenance dose is administered during the critical phase and/or recovery phase of the infection. In some embodiments, the maintenance dose is administered until the subject experiences a reduction in the one or more symptoms or until the one or more symptoms resolve.

In some embodiments, the pharmaceutical composition is suitable for oral administration. In some embodiments, the pharmaceutical composition comprises a solid oral dosage formulation. In some embodiments, the solid oral dosage formulation is a tablet. In some embodiments, the solid oral dosage formulation is a capsule. In some embodiments, the solid oral dosage formulation is provided in a blister pack container. In some embodiments, the pharmaceutical composition comprises an oral suspension. In some embodiments, the pharmaceutical composition comprises a liquid solution.

In some embodiments, the method further comprises administering a second pharmaceutical composition comprising an active agent or a pharmaceutically acceptable salt thereof, wherein the active agent is not etoricoxib. In some embodiments, the active agent or a pharmaceutically acceptable salt thereof is acetaminophen (paracetamol).

In some embodiments, the method does not increase the risk of an adverse event as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the method does not increase the rate of an adverse event as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the method results in fewer adverse events as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis. In some embodiments, the administration of etoricoxib results in greater pain reduction as compared to administration of acetaminophen to the subject. In some embodiments, the acetaminophen is administered as a dose of about 1 g/day, about 2 g/day, about 3 g/day, about 4 g/day, or about 5 g/day.

In certain aspects, the invention provides a method for treating a subject infected with a virus capable of causing Viral Hemorrhagic Fever (VHF), the method comprising: assessing the subject for one or more symptoms of VHF, and if one or more symptoms of VHF is present, administering to the subject a pharmaceutical composition comprising a therapeutically effective amount of etoricoxib or a pharmaceutically acceptable salt thereof.

In some embodiments, the method further comprises: reassessing symptoms following the administration of the pharmaceutical composition; and adjusting treatment according to the reassessed symptoms, wherein the adjusting comprises administering further the pharmaceutical composition if the reassessed symptoms are worse, not improved or improved but not gone compared to the assessed symptoms and terminating administering the pharmaceutical composition if the reassessed symptoms are gone compared to the assessed symptoms.

In some embodiments, the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

In some embodiments, the VHF is caused by one or more RNA viruses derived from a family of viruses selected from Arenaviridae, Bunyaviridae, Filoviridae, Flaviviridae, and any combinations thereof. In some embodiments, the VHF is caused by one or more RNA viruses from the Flaviviridae family of viruses. In some embodiments, the VHF is caused by a Dengue Virus.

In some embodiments, the therapeutically effective amount of the etoricoxib or a pharmaceutically acceptable salt thereof causes a reduction in viral load, viral titer, or viral shedding. In some embodiments, the pharmaceutical composition is administered at a duration and a dosage such that the subject experiences a reduction in the one or more symptoms. In some embodiments, the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

In some embodiments, the therapeutically effective amount of the etoricoxib or a pharmaceutically acceptable salt thereof is provided in a quantity sufficient for a single course of therapy. In some embodiments, the pharmaceutical composition is provided in a single course of therapy package.

In some embodiments, the pharmaceutical composition is administered at a dosing schedule matched to the time course of the VHF. In some embodiments, the pharmaceutical composition comprises a dose of etoricoxib sufficient to achieve a maximum anti-pyretic effect during the febrile phase of the infection. In some embodiments, the method further comprises administering a lower maintenance dose during the critical phase and/or recovery phase of the infection compared to the dose administered during the febrile phase of the infection. In some embodiments, the pharmaceutical composition is administered at a dose that does not affect platelet aggregation or bleeding time.

In some embodiments, the pharmaceutical composition is administered at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

In some embodiments, the pharmaceutical composition is administered as an initial loading dose of about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day. In some embodiments, the initial loading dose is administered during the febrile phase of the infection.

In some embodiments, the method further comprises administering a maintenance dose of about 30 mg/day, about 40 mg/day, about 50 mg/day about 60 mg/day, about 70 mg/day, about 80 mg/day, about 90 mg/day, about 100 mg/day, about 110 mg/day, or about 120 mg/day. In some embodiments, the maintenance dose is administered for up to 3 days, up to 5 days, up to 7 days, up to 10 days, or up to 14 days. In some embodiments, the maintenance dose is administered during the critical phase and/or recovery phase of the infection. In some embodiments, the maintenance dose is administered until the subject experiences a reduction in the one or more symptoms or until the one or more symptoms resolve.

In some embodiments, the pharmaceutical composition is suitable for oral administration. In some embodiments, the pharmaceutical composition comprises a solid oral dosage formulation. In some embodiments, the solid oral dosage formulation is a tablet. In some embodiments, the solid oral dosage formulation is a capsule. In some embodiments, the solid oral dosage formulation is provided in a blister pack container. In some embodiments, the pharmaceutical composition comprises an oral suspension formulation. In some embodiments, the pharmaceutical composition comprises a liquid solution formulation.

In some embodiments, the method further comprises a second pharmaceutical composition comprising an active agent or a pharmaceutically acceptable salt thereof, wherein the active agent is not etoricoxib. In some embodiments, the active agent or a pharmaceutically acceptable salt thereof is acetaminophen (paracetamol).

In some embodiments, the method does not increase the risk of an adverse event as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the method does not increase the rate of adverse events as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the method results in fewer adverse events as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the administration of etoricoxib results in greater pain reduction as compared to administration of acetaminophen to the subject. In some embodiments, the acetaminophen is administered as a dose of about 1 g/day, about 2 g/day, about 3 g/day, about 4 g/day, or about 5 g/day.

In certain aspects, the invention provides a method for treating a subject infected with a virus capable of causing Viral Hemorrhagic Fever (VHF), the method comprising: administering to the subject a pharmaceutical composition comprising an active agent or a pharmaceutically acceptable salt thereof at a sufficient dose to cause a reduction in viral load, viral titer, or viral shedding of the virus.

In some embodiments, the active agent is etoricoxib and wherein the pharmaceutical composition further comprises a pharmaceutically acceptable carrier.

In some embodiments, the VHF is caused by one or more RNA viruses derived from a family of viruses selected from Arenaviridae, Bunyaviridae, Filoviridae, Flaviviridae, and any combinations thereof. In some embodiments, the VHF is caused by one or more RNA viruses from the Flaviviridae family of viruses. In some embodiments, the VHF is caused by a Dengue Virus.

In some embodiments, the pharmaceutical composition is provided in a quantity sufficient for a single course of therapy. In some embodiments, the pharmaceutical composition is provided in a single course of therapy package. In some embodiments, the pharmaceutical composition is administered at a dosing schedule matched to the time course of the VHF. In some embodiments, the pharmaceutical composition comprises a dose of etoricoxib sufficient to achieve a maximum anti-pyretic effect during the febrile phase of an infection.

In some embodiments, the method further comprises administering a lower maintenance dose during the critical phase and/or recovery phase of the infection compared to the dose administered during the febrile phase of the infection. In some embodiments, the pharmaceutical composition is administered at a dose that does not affect platelet aggregation or bleeding time.

In some embodiments, the pharmaceutical composition is administered at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

In some embodiments, the pharmaceutical composition is administered as an initial loading dose of about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day. In some embodiments, the initial loading dose is administered during the febrile phase of the infection.

In some embodiments, the method further comprises administering a maintenance dose of about 30 mg/day, about 40 mg/day, about 50 mg/day about 60 mg/day, about 70 mg/day, about 80 mg/day, about 90 mg/day, about 100 mg/day, about 110 mg/day, or about 120 mg/day. In some embodiments, the maintenance dose is administered for up to 3 days, up to 5 days, up to 7 days, up to 10 days, or up to 14 days. In some embodiments, the maintenance dose is administered during the critical phase and/or recovery phase of the infection.

In some embodiments, the pharmaceutical composition is suitable for oral administration. In some embodiments, the pharmaceutical composition comprises a solid oral dosage formulation. In some embodiments, the solid oral dosage formulation is a tablet. In some embodiments, the solid oral dosage formulation is a capsule. In some embodiments, the solid oral dosage is provided in a blister pack container. In some embodiments, the pharmaceutical composition comprises an oral suspension formulation. In some embodiments, the pharmaceutical composition comprises a liquid solution formulation.

In some embodiments, the method further comprises administering a second pharmaceutical composition comprising an active agent or a pharmaceutically acceptable salt thereof, wherein the active agent is not etoricoxib. In some embodiments, the active agent is acetaminophen (paracetamol).

In some embodiments, the method does not increase the risk of an adverse event as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the method does not increase the rate of adverse events as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the method results in fewer adverse events as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the administration of etoricoxib results in greater pain reduction as compared to administration of acetaminophen to the subject. In some embodiments, the acetaminophen is administered as a dose of about 1 g/day, about 2 g/day, about 3 g/day, about 4 g/day, or about 5 g/day.

In certain aspects, the invention provides a method for treating a subject having Dengue fever, the method comprising: administering to the subject a pharmaceutical composition comprising a therapeutically effective amount of etoricoxib or a pharmaceutically acceptable salt thereof.

In some embodiments, the therapeutically effective amount of the etoricoxib or a pharmaceutically acceptable salt thereof causes a reduction in Dengue fever viral load, viral titer, or viral shedding. In some embodiments, the pharmaceutical composition is administered at a duration and a dosage such that the subject experiences a reduction in one or more Dengue fever symptoms. In some embodiments, the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

In some embodiments, the therapeutically effective amount of the etoricoxib of pharmaceutically acceptable salt thereof is provided in a quantity sufficient for a single course of therapy. In some embodiments, the therapeutically effective amount of the etoricoxib or a pharmaceutically acceptable salt thereof is provided in a single course of therapy package.

In some embodiments, the therapeutically effective amount of the etoricoxib or a pharmaceutically acceptable salt thereof is administered at a dosing schedule matched to the time course of the Dengue fever. In some embodiments, the pharmaceutical composition comprises a dose of etoricoxib sufficient to achieve maximum anti-pyretic effect during the febrile phase of the infection. In some embodiments, the method further comprises administering a lower maintenance dose during the critical phase and/or recovery phase of the infection compared to the dose administered during the febrile phase of the infection. In some embodiments, the pharmaceutical composition is administered at a dose that does not affect platelet aggregation or bleeding time.

In some embodiments, the pharmaceutical composition is administered at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

In some embodiments, the pharmaceutical composition is administered as an initial loading dose of about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day. In some embodiments, the initial loading dose is administered during the febrile phase of the infection.

In some embodiments, the method further comprises administering a maintenance dose of about 30 mg/day, about 40 mg/day, about 50 mg/day about 60 mg/day, about 70 mg/day, about 80 mg/day, about 90 mg/day, about 100 mg/day, about 110 mg/day, or about 120 mg/day. In some embodiments, the maintenance dose is administered for up to 3 days, up to 5 days, up to 7 days, up to 10 days, or up to 14 days. In some embodiments, the maintenance dose is administered during the critical phase and/or recovery phase of the infection. In some embodiments, the maintenance dose is administered until the subject experiences a reduction in one or more symptoms associated with Dengue fever or until the one or more symptoms resolve.

In some embodiments, the pharmaceutical composition is suitable for oral administration. In some embodiments, the pharmaceutical composition comprises a solid oral dosage formulation. In some embodiments, the solid oral dosage formulation is a tablet. In some embodiments, the solid oral dosage formulation is a capsule. In some embodiments, the solid oral dosage formulation is provided in a blister pack container. In some embodiments, the pharmaceutical composition comprises an oral suspension formulation. In some embodiments, the pharmaceutical composition comprises a liquid solution formulation.

In some embodiments, the method further comprises administering a second pharmaceutical composition comprising an active agent or a pharmaceutically acceptable salt thereof, wherein the active agent is not etoricoxib. In some embodiments, the active agent is acetaminophen (paracetamol).

In some embodiments, the method does not increase the risk of an adverse event as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the method does not increase the rate of adverse events as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the method results in fewer adverse events as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the administration of etoricoxib results in greater pain reduction as compared to administration of acetaminophen to the subject. In some embodiments, the acetaminophen is administered as a dose of about 1 g/day, about 2 g/day, about 3 g/day, about 4 g/day, or about 5 g/day.

In certain aspects, the invention provides a product for short-term use of etoricoxib by a subject in need thereof, the product comprising a single course therapy of etoricoxib or a pharmaceutically acceptable salt thereof.

In some embodiments, the product further comprises a container having a pre-defined number of solid oral dosage forms of the etoricoxib or a pharmaceutically acceptable salt thereof. In some embodiments, the solid oral dosage forms further comprise a pharmaceutically acceptable carrier.

In some embodiments, the single course therapy of etoricoxib or a pharmaceutically acceptable salt thereof comprises about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

In some embodiments, the single course therapy of etoricoxib or a pharmaceutically acceptable salt thereof comprises an initial loading dose of about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day, followed by a maintenance dose of etoricoxib of about 30 mg/day, about 40 mg/day, about 50 mg/day about 60 mg/day, about 70 mg/day, about 80 mg/day, about 90 mg/day, about 100 mg/day, about 110 mg/day, or about 120 mg/day. In some embodiments, the maintenance dose is provided for up to 3 days, up to 5 days, up to 7 days, up to 10 days, or up to 14 days.

In some embodiments, the solid oral dosage form is a tablet. In some embodiments, the solid oral dosage form is a capsule. In some embodiments, the solid oral dosage form is provided in a blister pack container.

In some embodiments, the therapeutically effective amount of the etoricoxib or a pharmaceutically acceptable salt thereof is 60 mg, 90 mg, or 120 mg.

### DETAILED DESCRIPTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

As used herein, “etoricoxib” means 5-chloro-2-(6-methylpyridin-3-yl)-3-[4-(trideuteriomethylsulfonyl)phenyl]pyridine. The term “etoricoxib” as used herein is inclusive of any pharmaceutically acceptable salts, hydrates, or solvates thereof; any crystalline form; and/or isotopically-enriched forms thereof, including deuterium-enriched forms. Etoricoxib, and a method of manufacturing etoricoxib, are described in U.S. Pat. No. 5,861,419, which is incorporated herein by reference in its entirety.

As used herein, the term “subject” refers to a vertebrate animal. In one embodiment, the subject is a mammal or a mammalian species. In one embodiment, the subject is a human. In other embodiments, the subject is a non-human vertebrate animal, including, without limitation, non-human primates, laboratory animals, livestock, racehorses, domesticated animals, and non-domesticated animals. The term “mammal” includes, but is not limited to, a human, mouse, rat, guinea pig, dog, cat, horse, cow, pig, or non-human primate, such as a monkey, chimpanzee, baboon or rhesus. In one embodiment, the mammal is a human.

As used herein, the term “patient” refers to a human or animal.

As used herein, “administered in combination” means the administration of two agents (e.g., concomitantly or sequentially) in any manner in which the pharmacological effects of both are manifest in the subject at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

In certain aspects, the invention provides a method for treating one or more symptoms in a subject having viral hemorrhagic fever (VHF), the method comprising administering to the subject a pharmaceutical composition comprising a therapeutically effective amount of etoricoxib or a pharmaceutically acceptable salt thereof.

In some embodiments, the pharmaceutical composition further comprises a pharmaceutically acceptable carrier. In some embodiments, the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

In some embodiments, the VHF is caused by one or more RNA viruses derived from a family of viruses selected from Arenaviridae, Bunyaviridae, Filoviridae, Flaviviridae, and any combinations thereof. In some embodiments, the VHF is caused by one or more RNA viruses from the Flaviviridae family of viruses. In some embodiments, the VHF is caused by a Dengue Virus.

In some embodiments, the therapeutically effective amount of the etoricoxib causes a reduction in viral load, viral titer, or viral shedding. In some embodiments, the pharmaceutical composition is administered at a duration and a dosage such that the subject experiences a reduction in the one or more symptoms. In some embodiments, the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

In some embodiments, the pharmaceutical composition is provided in a quantity sufficient for a single course of therapy. In some embodiments, the pharmaceutical composition is provided in a single course of therapy package.

In some embodiments, the pharmaceutical composition is administered at a dosing schedule matched to the time course of the VHF. In some embodiments, the pharmaceutical composition comprises a dose of etoricoxib sufficient to achieve maximum anti-pyretic effect during the febrile phase of the infection. In some embodiments, the method further comprises administering to the subject a lower maintenance dose during the critical phase and/or recovery phase of the infection compared to the dose administered during the febrile phase of the infection. In some embodiments, the pharmaceutical composition comprising etoricoxib is administered at a dose that does not affect platelet aggregation or bleeding time.

In some embodiments, the pharmaceutical composition is administered at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

In some embodiments, the pharmaceutical composition is administered as an initial loading dose of about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day. In some embodiments, the initial loading dose is administered during the febrile phase of the infection.

In some embodiments, the method further comprises administering a maintenance dose of etoricoxib at about 30 mg/day, about 40 mg/day, about 50 mg/day about 60 mg/day, about 70 mg/day, about 80 mg/day, about 90 mg/day, about 100 mg/day, about 110 mg/day, or about 120 mg/day. In some embodiments, the maintenance dose is administered for up to 3 days, up to 5 days, up to 7 days, up to 10 days, or up to 14 days. In some embodiments, the maintenance dose is administered during the critical phase and/or recovery phase of the infection. In some embodiments, the maintenance dose is administered until the subject experiences a reduction in the one or more symptoms or until the one or more symptoms resolve.

In some embodiments, the pharmaceutical composition is suitable for oral administration. In some embodiments, the pharmaceutical composition comprises a solid oral dosage formulation. In some embodiments, the solid oral dosage formulation is a tablet. In some embodiments, the solid oral dosage formulation is a capsule. In some embodiments, the solid oral dosage formulation is provided in a blister pack container. In some embodiments, the pharmaceutical composition comprises an oral suspension. In some embodiments, the pharmaceutical composition comprises a liquid solution.

In some embodiments, the method further comprises administering a second pharmaceutical composition comprising an active agent or a pharmaceutically acceptable salt thereof, wherein the active agent is not etoricoxib. In some embodiments, the active agent or a pharmaceutically acceptable salt thereof is acetaminophen (paracetamol).

In some embodiments, the method does not increase the risk of an adverse event as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the method does not increase the rate of an adverse event as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the method results in fewer adverse events as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis. In some embodiments, the administration of etoricoxib results in greater pain reduction as compared to administration of acetaminophen to the subject. In some embodiments, the acetaminophen is administered as a dose of about 1 g/day, about 2 g/day, about 3 g/day, about 4 g/day, or about 5 g/day.

In certain aspects, the invention provides a method for treating a subject infected with a virus capable of causing Viral Hemorrhagic Fever (VHF), the method comprising: assessing the subject for one or more symptoms of VHF, and if one or more symptoms of VHF is present, administering to the subject a pharmaceutical composition comprising a therapeutically effective amount of etoricoxib or a pharmaceutically acceptable salt thereof.

In some embodiments, the method further comprises: reassessing symptoms following the administration of the pharmaceutical composition; and adjusting treatment according to the reassessed symptoms, wherein the adjusting comprises administering further the pharmaceutical composition if the reassessed symptoms are worse, not improved or improved but not gone compared to the assessed symptoms and terminating administering the pharmaceutical composition if the reassessed symptoms are gone compared to the assessed symptoms.

In some embodiments, the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

In some embodiments, the VHF is caused by one or more RNA viruses derived from a family of viruses selected from Arenaviridae, Bunyaviridae, Filoviridae, Flaviviridae, and any combinations thereof. In some embodiments, the VHF is caused by one or more RNA viruses from the Flaviviridae family of viruses. In some embodiments, the VHF is caused by a Dengue Virus.

In some embodiments, the therapeutically effective amount of the etoricoxib or a pharmaceutically acceptable salt thereof causes a reduction in viral load, viral titer, or viral shedding. In some embodiments, the pharmaceutical composition is administered at a duration and a dosage such that the subject experiences a reduction in the one or more symptoms. In some embodiments, the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

In some embodiments, the therapeutically effective amount of the etoricoxib or a pharmaceutically acceptable salt thereof is provided in a quantity sufficient for a single course of therapy. In some embodiments, the pharmaceutical composition is provided in a single course of therapy package.

In some embodiments, the pharmaceutical composition is administered at a dosing schedule matched to the time course of the VHF. In some embodiments, the pharmaceutical composition comprises a dose of etoricoxib sufficient to achieve a maximum anti-pyretic effect during the febrile phase of the infection. In some embodiments, the method further comprises administering a lower maintenance dose during the critical phase and/or recovery phase of the infection compared to the dose administered during the febrile phase of the infection. In some embodiments, the pharmaceutical composition is administered at a dose that does not affect platelet aggregation or bleeding time.

In some embodiments, the pharmaceutical composition is administered at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

In some embodiments, the pharmaceutical composition is administered as an initial loading dose of about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day. In some embodiments, the initial loading dose is administered during the febrile phase of the infection.

In some embodiments, the method further comprises administering a maintenance dose of about 30 mg/day, about 40 mg/day, about 50 mg/day about 60 mg/day, about 70 mg/day, about 80 mg/day, about 90 mg/day, about 100 mg/day, about 110 mg/day, or about 120 mg/day. In some embodiments, the maintenance dose is administered for up to 3 days, up to 5 days, up to 7 days, up to 10 days, or up to 14 days. In some embodiments, the maintenance dose is administered during the critical phase and/or recovery phase of the infection. In some embodiments, the maintenance dose is administered until the subject experiences a reduction in the one or more symptoms or until the one or more symptoms resolve.

In some embodiments, the pharmaceutical composition is suitable for oral administration. In some embodiments, the pharmaceutical composition comprises a solid oral dosage formulation. In some embodiments, the solid oral dosage formulation is a tablet. In some embodiments, the solid oral dosage formulation is a capsule. In some embodiments, the solid oral dosage formulation is provided in a blister pack container. In some embodiments, the pharmaceutical composition comprises an oral suspension formulation. In some embodiments, the pharmaceutical composition comprises a liquid solution formulation.

In some embodiments, the method further comprises a second pharmaceutical composition comprising an active agent or a pharmaceutically acceptable salt thereof, wherein the active agent is not etoricoxib. In some embodiments, the active agent or a pharmaceutically acceptable salt thereof is acetaminophen (paracetamol).

In some embodiments, the method does not increase the risk of an adverse event as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the method does not increase the rate of adverse events as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the method results in fewer adverse events as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the administration of etoricoxib results in greater pain reduction as compared to administration of acetaminophen to the subject. In some embodiments, the acetaminophen is administered as a dose of about 1 g/day, about 2 g/day, about 3 g/day, about 4 g/day, or about 5 g/day.

In certain aspects, the invention provides a method for treating a subject infected with a virus capable of causing Viral Hemorrhagic Fever (VHF), the method comprising: administering to the subject a pharmaceutical composition comprising an active agent or a pharmaceutically acceptable salt thereof at a sufficient dose to cause a reduction in viral load, viral titer, or viral shedding of the virus.

In some embodiments, the active agent is etoricoxib and wherein the pharmaceutical composition further comprises a pharmaceutically acceptable carrier.

In some embodiments, the VHF is caused by one or more RNA viruses derived from a family of viruses selected from Arenaviridae, Bunyaviridae, Filoviridae, Flaviviridae, and any combinations thereof. In some embodiments, the VHF is caused by one or more RNA viruses from the Flaviviridae family of viruses. In some embodiments, the VHF is caused by a Dengue Virus.

In some embodiments, the pharmaceutical composition is provided in a quantity sufficient for a single course of therapy. In some embodiments, the pharmaceutical composition is provided in a single course of therapy package. In some embodiments, the pharmaceutical composition is administered at a dosing schedule matched to the time course of the VHF. In some embodiments, the pharmaceutical composition comprises a dose of etoricoxib sufficient to achieve a maximum anti-pyretic effect during the febrile phase of an infection.

In some embodiments, the method further comprises administering a lower maintenance dose during the critical phase and/or recovery phase of the infection compared to the dose administered during the febrile phase of the infection. In some embodiments, the pharmaceutical composition is administered at a dose that does not affect platelet aggregation or bleeding time.

In some embodiments, the pharmaceutical composition is administered at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

In some embodiments, the pharmaceutical composition is administered as an initial loading dose of about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day. In some embodiments, the initial loading dose is administered during the febrile phase of the infection.

In some embodiments, the method further comprises administering a maintenance dose of about 30 mg/day, about 40 mg/day, about 50 mg/day about 60 mg/day, about 70 mg/day, about 80 mg/day, about 90 mg/day, about 100 mg/day, about 110 mg/day, or about 120 mg/day. In some embodiments, the maintenance dose is administered for up to 3 days, up to 5 days, up to 7 days, up to 10 days, or up to 14 days. In some embodiments, the maintenance dose is administered during the critical phase and/or recovery phase of the infection.

In some embodiments, the pharmaceutical composition is suitable for oral administration. In some embodiments, the pharmaceutical composition comprises a solid oral dosage formulation. In some embodiments, the solid oral dosage formulation is a tablet. In some embodiments, the solid oral dosage formulation is a capsule. In some embodiments, the solid oral dosage is provided in a blister pack container. In some embodiments, the pharmaceutical composition comprises an oral suspension formulation. In some embodiments, the pharmaceutical composition comprises a liquid solution formulation.

In some embodiments, the method further comprises administering a second pharmaceutical composition comprising an active agent or a pharmaceutically acceptable salt thereof, wherein the active agent is not etoricoxib. In some embodiments, the active agent is acetaminophen (paracetamol).

In some embodiments, the method does not increase the risk of an adverse event as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the method does not increase the rate of adverse events as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the method results in fewer adverse events as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the administration of etoricoxib results in greater pain reduction as compared to administration of acetaminophen to the subject. In some embodiments, the acetaminophen is administered as a dose of about 1 g/day, about 2 g/day, about 3 g/day, about 4 g/day, or about 5 g/day.

In certain aspects, the invention provides a method for treating a subject having Dengue fever, the method comprising: administering to the subject a pharmaceutical composition comprising a therapeutically effective amount of etoricoxib or a pharmaceutically acceptable salt thereof.

In some embodiments, the therapeutically effective amount of the etoricoxib or a pharmaceutically acceptable salt thereof causes a reduction in Dengue fever viral load, viral titer, or viral shedding. In some embodiments, the pharmaceutical composition is administered at a duration and a dosage such that the subject experiences a reduction in one or more Dengue fever symptoms. In some embodiments, the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

In some embodiments, the therapeutically effective amount of the etoricoxib of pharmaceutically acceptable salt thereof is provided in a quantity sufficient for a single course of therapy. In some embodiments, the therapeutically effective amount of the etoricoxib or a pharmaceutically acceptable salt thereof is provided in a single course of therapy package.

In some embodiments, the therapeutically effective amount of the etoricoxib or a pharmaceutically acceptable salt thereof is administered at a dosing schedule matched to the time course of the Dengue fever. In some embodiments, the pharmaceutical composition comprises a dose of etoricoxib sufficient to achieve maximum anti-pyretic effect during the febrile phase of the infection. In some embodiments, the method further comprises administering a lower maintenance dose during the critical phase and/or recovery phase of the infection compared to the dose administered during the febrile phase of the infection. In some embodiments, the pharmaceutical composition is administered at a dose that does not affect platelet aggregation or bleeding time.

In some embodiments, the pharmaceutical composition is administered at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

In some embodiments, the pharmaceutical composition is administered as an initial loading dose of about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day. In some embodiments, the initial loading dose is administered during the febrile phase of the infection.

In some embodiments, the method further comprises administering a maintenance dose of about 30 mg/day, about 40 mg/day, about 50 mg/day about 60 mg/day, about 70 mg/day, about 80 mg/day, about 90 mg/day, about 100 mg/day, about 110 mg/day, or about 120 mg/day. In some embodiments, the maintenance dose is administered for up to 3 days, up to 5 days, up to 7 days, up to 10 days, or up to 14 days. In some embodiments, the maintenance dose is administered during the critical phase and/or recovery phase of the infection. In some embodiments, the maintenance dose is administered until the subject experiences a reduction in one or more symptoms associated with Dengue fever or until the one or more symptoms resolve.

In some embodiments, the pharmaceutical composition is suitable for oral administration. In some embodiments, the pharmaceutical composition comprises a solid oral dosage formulation. In some embodiments, the solid oral dosage formulation is a tablet. In some embodiments, the solid oral dosage formulation is a capsule. In some embodiments, the solid oral dosage formulation is provided in a blister pack container. In some embodiments, the pharmaceutical composition comprises an oral suspension formulation. In some embodiments, the pharmaceutical composition comprises a liquid solution formulation.

In some embodiments, the method further comprises administering a second pharmaceutical composition comprising an active agent or a pharmaceutically acceptable salt thereof, wherein the active agent is not etoricoxib. In some embodiments, the active agent is acetaminophen (paracetamol).

In some embodiments, the method does not increase the risk of an adverse event as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the method does not increase the rate of adverse events as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the method results in fewer adverse events as compared to administration of acetaminophen to the subject. In some embodiments, the adverse event is a bleeding event. In some embodiments, the bleeding event is gastrointestinal bleeding. In some embodiments, the adverse event is an adverse cardiovascular event. In some embodiments, the adverse cardiovascular event is cardiothrombosis.

In some embodiments, the administration of etoricoxib results in greater pain reduction as compared to administration of acetaminophen to the subject. In some embodiments, the acetaminophen is administered as a dose of about 1 g/day, about 2 g/day, about 3 g/day, about 4 g/day, or about 5 g/day.

In certain aspects, the invention provides a product for short-term use of etoricoxib by a subject in need thereof, the product comprising a single course therapy of etoricoxib or a pharmaceutically acceptable salt thereof.

In some embodiments, the product further comprises a container having a pre-defined number of solid oral dosage forms of the etoricoxib or a pharmaceutically acceptable salt thereof. In some embodiments, the solid oral dosage forms further comprise a pharmaceutically acceptable carrier.

In some embodiments, the single course therapy of etoricoxib or a pharmaceutically acceptable salt thereof comprises about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

In some embodiments, the single course therapy of etoricoxib or a pharmaceutically acceptable salt thereof comprises an initial loading dose of about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day, followed by a maintenance dose of etoricoxib of about 30 mg/day, about 40 mg/day, about 50 mg/day about 60 mg/day, about 70 mg/day, about 80 mg/day, about 90 mg/day, about 100 mg/day, about 110 mg/day, or about 120 mg/day. In some embodiments, the maintenance dose is provided for up to 3 days, up to 5 days, up to 7 days, up to 10 days, or up to 14 days.

In some embodiments, the solid oral dosage form is a tablet. In some embodiments, the solid oral dosage form is a capsule. In some embodiments, the solid oral dosage form is provided in a blister pack container.

In some embodiments, the therapeutically effective amount of the etoricoxib or a pharmaceutically acceptable salt thereof is 60 mg, 90 mg, or 120 mg.

Clinical Description of VHFs

Though diverse in the underlying pathogens, common symptoms of VHFs include fever, myalgia, arthralgia, nausea, vomiting, and prostration. Physical examination may reveal only conjunctival injection, mild hypotension, flushing and petechial hemorrhages. Severe VHF typically evolves to shock and generalized mucous membrane hemorrhage and often is accompanied by evidence of pulmonary hematopoietic and neurologic involvement. Renal insufficiency is proportional to cardiovascular compromise, except in HFRS, which features renal failure as an integral part of the disease process.

The clinical syndrome that these viruses may cause is VHF, however, this syndrome is variable in its presentation. Bleeding may be an uncommon feature and not very impressive when it occurs (as in mild forms of Dengue fever or Rift Valley fever) or it may present as a copious life-threatening hemorrhage, as in Crimean Congo hemorrhagic fever. The progression to a septic shock-like state may be due to a combination of increases in vascular permeability, vasodilation, decreased myocardial function and fluid loss.

VHF should be suspected in any patient presenting with a severe febrile illness and evidence of vascular involvement (postural hypotension, petechiae, easy bleeding, flushing of face and chest, non-dependent edema), who has traveled to an area where the virus is known to occur, or where infectious disease surveillance suggests an outbreak. Symptoms and signs suggesting additional organ system involvement are common (e.g., headache, photophobia, pharyngitis, cough, nausea or vomiting, diarrhea, constipation, abdominal pain, hyperesthesia, dizziness, confusion, tremor), but usually do not dominate the clinical presentation. A positive tourniquet test has been particularly useful in Dengue hemorrhagic fever, but should be sought in other hemorrhagic fevers as well.

Not all infected patients with viruses capable of causing VHF end up developing VHF. There is both divergence and uncertainty about which host factors and viral strain characteristics might be responsible for the mechanisms of disease. For example, an immunopathogenic mechanism has been identified for Dengue fever, which usually occurs among patients previously infected with a heterologous Dengue serotype. Antibodies directed against the previous strain enhances uptake of the Dengue virus by circulating monocytes. These cells express viral antigens on their surfaces. Lysis of the infected monocytes by cytotoxic T-cell responses results in the release of pro-inflammatory cytokines, pro-coagulants and anticoagulants, which in turn results in vascular injury and permeability, complement activation and a systemic coagulopathy.

Diffuse or disseminated intravascular coagulation (DIC) has been implicated in Rift Valley, Marburg and Ebola fevers, but in most VHFs the etiology of the coagulopathy is multifactorial (e.g., hepatic damage, consumptive coagulopathy and primary marrow injury to megakaryocytes).

Treatment of VHFs

There are currently no approved curative treatments for VHFs, though some antiviral therapies, such as, for example, ribavirin, have demonstrated some utility in treating several of these infections. Other preventative or curative treatments for VHFs are under investigation. The primary treatments for patients with VHFs are supportive care to address acute symptom manifestation and, depending on the severity of symptoms and underlying health of the infected patient, intensive supportive care as required.

General principles of supportive care apply to hemodynamic, hematologic, pulmonary and neurologic manifestations of VHF, regardless of the specific etiologic agent. These patients all require intensive care, but the effectiveness of supportive care leads to variable outcomes depending on etiology of disease (i.e., very good in Dengue fever, but not very effective in Yellow Fever). Management of these diseases is similar to the management of septic shock.

Healthcare providers employing vigorous fluid resuscitation of hypotensive patients must be mindful of the propensity of some VHFs (e.g., HFRS) for pulmonary capillary leak. Pressor agents are frequently required to address hypotension. The use of intravascular devices and invasive hemodynamic monitoring must be carefully considered in the context of potential benefit versus the risk of hemorrhage. Restlessness, confusion, myalgia, and hyperesthesia should be managed by conservative measures, and the judicious use of sedatives and analgesics/anti-pyretics such as, for example, acetaminophen. Secondary infections may occur as with any patient undergoing intensive care utilizing invasive procedures and devices, such as intravenous lines and indwelling catheters.

The management of clinical bleeding should follow the same principles as for any patient with a systemic coagulopathy and assisted by coagulation studies. It has been recommended, therefore, that intramuscular injections, aspirin or any NSAIDs (e.g., ibuprofen, celecoxib) and other anticoagulant drugs should be avoided.

NSAIDs

The over expression of cyclooxygenase-2 (COX-2) plays a major role in the replication of several types of viruses, including Hepatitis C and Dengue, and it was once hypothesized that the inhibition of COX-2 via COX-2 inhibitors such as traditional NSAIDs or selective COX-2 inhibitors could serve as a treatment option in certain viral infections. However, due to the confounding risk of exacerbating gastritis and hemorrhage in patients infected with VHF such as the Dengue virus, use of NSAIDs and COX-2 selective NSAIDs (e.g., celecoxib) have not been recommended to treat the symptoms of Dengue fever. In fact, healthcare authorities and organizations such as the World Health Organization specifically instruct healthcare providers to avoid administering NSAIDs when treating patients with VHF.

Non-steroidal anti-inflammatory drugs (NSAIDs) are among the most widely used drugs in the world. They are chiefly used to treat inflammatory-related pain conditions, but their short-term use is limited because of the effects on bleeding time and platelet aggregation in situations with hemorrhagic risk, and their long-term use is limited by serious gastrointestinal side-effects. NSAIDs inhibit the two recognized forms of prostaglandin G/H synthase (also referred to as cyclo-oxygenase, or “COX”), namely COX-1 and COX-2. Since the analgesic, anti-pyretic, and anti-inflammatory effects of NSAIDs are mediated by inhibition of COX-2, and their platelet aggregation and gastrointestinal side effects mostly influenced by inhibition of COX-1, NSAIDs which selectively inhibit COX-2 could theoretically reduce the risk of hemorrhagic effects and gastrointestinal toxicity compared with other NSAIDs while still providing analgesic, anti-pyretic, and anti-inflammatory effects.

Based on this premise, several COX-2 selective NSAIDs, such as etoricoxib, were developed in the 1990s. Early trials comparing selective COX-2 NSAIDs versus non-selective NSAIDs, such as naproxen and ibuprofen, seemed to confirm that the selective COX-2 NSAIDs at doses with similar analgesic efficacy had less gastrointestinal toxicity. In addition, COX-2 selective NSAIDs demonstrated that they do not affect platelet thromboxane production and do not impair platelet function, unlike non-selective NSAIDs. However, placebo-controlled trials have shown unequivocally that COX-2 selective NSAIDs are associated with an increased risk of atherothrombotic vascular events. Subsequent to these findings, several COX-2 selective NSAIDs were promptly removed from the market and/or non-approved. Analyses also showed that the CV effects of traditional NSAIDs and COX-2 selective NSAIDs were dose and duration dependent, with the highest risk coming with chronic use of otherwise acute use doses.

Though later analyses have demonstrated that the cardiovascular (CV) effects of non-selective NSAIDs and COX-2 selective NSAIDs appear to be proportional to the magnitude of COX-2 inhibition and, therefore, equa-potent doses of non-selective NSAIDs appear to carry a similar CV risk profile to that of COX-2 selective NSAIDs, the use of COX-2 selective NSAIDs in general has fallen out of favor and their use is not generally recommended.

Finally, both non-selective NSAIDs and selective COX-2 NSAIDs have known anti-pyretic (e.g., fever reducing) properties. Though a pronounced and potentially useful pharmacological effect, it is believed that these anti-pyretic properties are a potential safety concern as, when used in the context of the treatment of acute or inflammatory pain, these products, in reducing fever, could mask the symptoms of an underlying and untreated infection.

Though all COX-2 selective NSAIDs share a common demonstration of little to no effect on bleeding time or anti-platelet inhibition at clinical doses used for pain, among the class there are wide differences on variables such as: selectivity for COX-2 isozyme, GI safety, half-live, onset of action, and dosing interval.

Etoricoxib

Etoricoxib is a COX-2 selective NSAID with potent anti-pyretic and anti-inflammatory properties. Etoricoxib is highly COX-2 selective, with a 106-fold COX-2 versus COX-1 selectivity. Unlike non-selective NSAIDs, etoricoxib, at doses up to 500 mg, does not affect platelet aggregation or bleeding time. Etoricoxib has also demonstrated significant improvements in GI tolerability and safety events versus non-selective NSAIDs.

Etoricoxib may be administered as an oral product which may be administered once daily, and has a half-live of approximately 22 hours. Etoricoxib has been shown to have rapid and sustained anti-inflammatory efficacy, with a median onset of action in acute pain settings of 24 minutes and sustained efficacy over 24 hours with a single dose.

Etoricoxib was rejected by the FDA in 2007 for approval in the United States due to safety concerns relevant in chronic use.

Problems Addressed by the Subject Matter Disclosed Herein

The cluster of viral hemorrhagic fevers (VHFs) and their related pseudo-similar diseases (e.g., Zika Virus), though diverse in their viral origins, all carry similar phenotypical symptoms and corresponding treatment related challenges. Each of the diseases manifest with rapid high-grade fever (pyrexia), pain, inflammation, nausea, vomiting, rash, and, except for Zika, varying degrees of potential hemorrhagic events. In addition, it is believed that disease morbidity and outcomes are correlated to the initial severity of the fever and initial viral levels. It is also believed that COX-2 expression may play a role in the replication of some types of viruses, including replication of the Dengue virus.

Due to concerns with exacerbation of potential bleeding risk and gastritis, health care authorities and organizations do not recommend using a known effective anti-pyretic and anti-inflammatory such as non-selective NSAIDs and COX-2 selective NSAIDs to address fever and inflammation associated with VHFs, but instead recommend the use of acetaminophen to address fever and pain. Not only are NSAIDs not mentioned or recommended as a treatment option, health care authorities and organizations explicitly teach against their use to treat VHFs due to their “platelet effects”. See “Clinical Management of Patients with Viral Hemorrhagic Fever: A Pocket Guide for the Front-line Health Worker,” World Health Organization (Mar. 30, 2014). Despite this guidance, acetaminophen's properties (i.e., no anti-inflammatory properties, requires frequent and large doses, significant dose-related hepatic toxicities) limits its utility in this role.

In addition to these limitations specific to VHFs, COX-2 selective NSAIDs in general are not recommended for treating pain, inflammation and fever due to concerns regarding cardiovascular safety. Though many believe this is an absolute risk, evidence indicates that CV risk with COX-2 selective NSAIDs is a dose and duration dependent effect.

It has been surprisingly discovered that the clinical limitations of certain COX-2 selective NSAIDs are not applicable to subjects suffering from an acute, short duration (often less than 14 days), high morbidity, potentially high mortality, and self-limiting condition, such as, for example, a VHF, and that the short-term use of etoricoxib can provide significant benefits to subjects suffering from VHFs, including Dengue fever.

In addition, as fever severity in subject with VHFs is correlated with disease morbidity and possibly outcomes, it has been surprisingly discovered that direct pharmacological intervention with etoricoxib, due in part to its potent anti-pyretic effect, for a short duration can address fever in these subjects in such a way to influence the outcome of the disease, while avoiding the toxicities associated with long-term use of these agents.

Finally, as it is believed that viral load of the Dengue virus is correlated with disease severity and that COX-2 inhibition could play a role in Dengue viral replication, it has been surprisingly discovered that direct pharmacological intervention with etoricoxib, a potent COX-2 selective NSAID with a long half-life and sustained inhibitory effect for a short duration may suppress COX-2 replication in these subjects, reduce disease severity, and minimize or avoid the toxicities associated with long-term use of COX-2 selective NSAIDs.

Described herein are methods and products for treating viral hemorrhagic fevers and related symptoms with etoricoxib.

It has been surprisingly discovered that etoricoxib may be effective in treating a viral hemorrhagic fever, including associated fever, pain and inflammation, in subjects infected with a virus capable of causing a viral hemorrhagic fever, such as Dengue fever. It has also been surprisingly discovered that etoricoxib may be safely used to treat a viral hemorrhagic fever, including associated fever, pain and inflammation, in a subject without increasing the risk of bleeding or other adverse platelet effects; gastritis or other gastrointestinal-related side effects or adverse events; or cardiovascular-related side effects or adverse events in the subject.

In one aspect, a method is provided for treating viral hemorrhagic fever in a subject, wherein the method comprises administering etoricoxib to the subject.

In certain aspects, a method is provided for treating viral hemorrhagic fever in a subject, wherein the method comprises administering a dose of 90 to 500 mg per day of etoricoxib to the subject. In some embodiments, the method comprises administering a dose of 90 to 500 mg per day of etoricoxib to the subject as an initial dose. In some embodiments, the initial dose is administered during the febrile phase of the infection. In some embodiments, the method further comprises administering a maintenance dose of 30 to 120 mg per day of etoricoxib to the subject. In some embodiments, the maintenance dose is administered after the febrile phase of the infection, or during the critical phase, the recovery phase, or both. In some embodiments, the treatment of the viral hemorrhagic fever comprises treating one or more symptoms of the viral hemorrhagic fever, such as fever, pain, or inflammation. In some embodiments, the maintenance dose is administered until the subject experiences a reduction in the one or more symptoms or until the one or more symptoms resolve.

In certain aspects, a method is provided for treating viral hemorrhagic fever in a subject, the method comprising administering to the subject etoricoxib in an amount sufficient to cause a reduction in the viral level or viral load of the virus causing the viral hemorrhagic fever in the subject. Viral load is also known as viral burden, viral titre or viral titer. Viral load is a numerical expression of the quantity of virus in a given volume. It is often expressed as particles per ml. Viral shedding is the release of virus progeny following successful reproduction during a host-cell infection. Once replication has been completed the viruses may begin to leave the cell using different methods.

In certain aspects, a method is provided for treating a subject infected with the Zika virus, the method comprising administering to the subject etoricoxib. In some embodiments the method comprises treating one or more symptoms of the Zika virus in the subject. In some embodiments, the one or more symptoms comprise pain, fever, or inflammation. In some embodiments, the method comprises administering etoricoxib in an amount sufficient to cause a reduction in the viral level of the Zika virus.

In certain aspects, a product is provided for the short-term use of etoricoxib through a single course of therapy. In some embodiments, the product comprises a container comprising a predefined number of solid dosage forms of a pharmaceutical composition comprising etoricoxib and a pharmaceutically acceptable carrier. In some embodiments, the solid dosage form is an oral tablet. In some embodiments, the container is a blister pack.

Without limiting the scope of this disclosure, in any of the methods described herein, the treating of the viral hemorrhagic fever may comprise the treating of one or more symptoms of the viral hemorrhagic fever, including fever, pain, or inflammation; where not specified, the viral hemorrhagic fever may be Dengue fever; the subject may be a human patient (adult or child); the pharmaceutical composition may be orally administered or suitable for oral administration, such as a solid dosage form (tablet or capsule) or oral suspension or liquid; the pharmaceutical composition may comprise between 10-20 mg, 20-40 mg, 40-60 mg, 60-80 mg, 80-100 mg, 100-120 mg, 120-140 mg, or more of etoricoxib; in some embodiments the pharmaceutical composition may comprise about 30 mg, 40 mg, 50 mg, 60 mg, 70 mg, 80 mg, 90 mg, 100 mg, 110 mg or 120 mg of etoricoxib for oral or intravenous administration; the method may comprise further administering acetaminophen to the subject; and the method may be performed without causing or increasing the risk of a bleeding event (including gastrointestinal bleeding) or adverse cardiovascular event (e.g., cardiothrombosis) in the subject.

Compositions

Etoricoxib is a nonsteroidal anti-inflammatory drug that exhibits anti-inflammatory, analgesic, and antipyretic activities. Without being bound by theory, the mechanism of action of etoricoxib is believed to be due to the selective inhibition of cyclooxygenase-2 (COX-2). The chemical structure of etoricoxib is shown below:

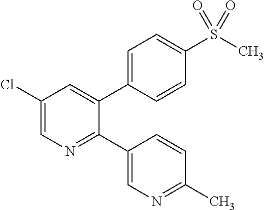 [SMILES: Cc1ccc(-c2ncc(Cl)cc2-c2ccc(S(C)(=O)=O)cc2)cn1]

Etoricoxib as described herein may be provided in or with a “pharmaceutically acceptable carrier” or “pharmaceutically acceptable excipient”, both of which are used interchangeably herein, to form a pharmaceutical composition. The pharmaceutically acceptable carrier may be selected on the basis of the desired route of administration of the compound.

Pharmaceutical compositions of etoricoxib which may be used in the methods or products described herein include those suitable for oral (e.g., tablet or capsule), intravenous, parenteral (e.g., subcutaneous, intramuscular, and intradermal injections, or infusion techniques), sublingual, or other administration, although the most suitable route in any given case will depend on the nature and severity of the condition being treated and on the nature of the particular compound which is being used.

Methods of Use

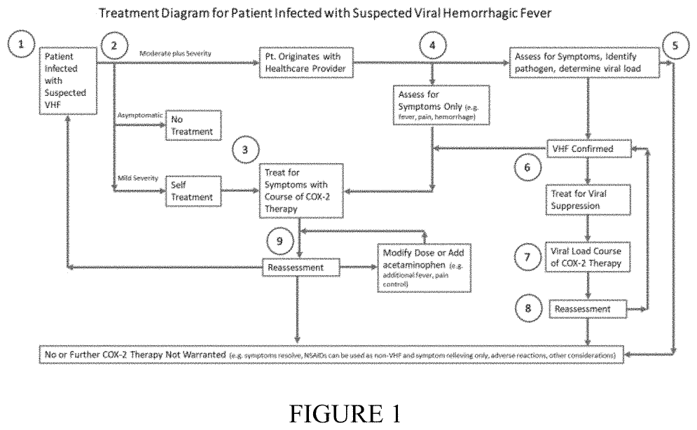

FIG. 1 illustrates one embodiment comprising a method of treating a subject infected with a virus capable of causing viral hemorrhagic fever by administering etoricoxib. In block 1, a subject becomes infected (e.g., via a bite from a mosquito, tick, contact with an infected person) with a virus. In block 2, depending on the severity of the symptoms, the subject may choose to do nothing if asymptomatic; self-treat if experiencing an infection of mild severity; or contact a healthcare provider seeking medical treatment if the symptoms are more severe or the subject is more physically compromised (e.g., elderly, child). In block 3, the subject is treated with a course of therapy with etoricoxib to address symptoms of the suspected viral hemorrhagic fever, either via self-administration, if direct access to etoricoxib by the subject is possible, or as directed by a healthcare provider. In block 4, the healthcare provider assesses the subject for symptoms to determine if a viral hemorrhagic fever is likely involved, and additionally may employ more sophisticated diagnostic techniques (e.g., identification of the viral pathogen via anti-body test or other methods, quantitative viral load assessment). In block 6, with a confirmed diagnosis of a viral hemorrhagic fever, the healthcare provider can choose to treat the symptoms only of the infection with etoricoxib, or choose to suppress viral replication. In block 7, the healthcare provider utilizes a dose and duration of treatment with etoricoxib designed to suppress viral replication of the now-identified pathogen. In block 8, after a course of treatment to suppress viral replication, the healthcare provider reassesses their treatment results and can pursue another course of therapy to suppress viral replication, treat for symptom relief only, or discontinue treatment with etoricoxib. In block 9, the healthcare provider, or self-treating subject, who is treating for symptom relief reassesses their treatment results and can modify the dose or duration of treatment with etoricoxib, add acetaminophen or another analgesic if additional fever reduction or pain relief is necessary and the dose limit of etoricoxib has been reached, self-refer to a healthcare provider if symptoms persist or worsen (in the case of a self-treating subject) or discontinue treatment with etoricoxib if conditions warrant.

In certain aspects, a method is provided for treating viral hemorrhagic fever in a subject, wherein the method comprises administering etoricoxib to the subject. In some embodiments, the method of treating viral hemorrhagic fever may comprise treating one or more symptoms of viral hemorrhagic fever, such as fever, pain, or inflammation. In some embodiments, in the treating the one or more symptoms of viral hemorrhagic fever, the method may comprise administering etoricoxib at a sufficient dose and duration to reduce the symptoms in the subject. In some embodiments, the method may comprise treating the subject to reduce the viral level of the virus causing the viral hemorrhagic fever in the subject. In some embodiments, in treating the subject to reduce the viral level of the virus, the method may comprise administering etoricoxib at a sufficient dose and duration to reduce the viral level of the virus. In some embodiments, the pharmaceutical composition may comprise a dosage form suitable for oral administration, such as a tablet, capsule, suspension or liquid, or a dosage form suitable for parenteral administration, such as a solution suitable for intravenous administration. In some embodiments, the method may comprise administering 20-40 mg, 40-60 mg, 60-80 mg, 80-100 mg, 100-120 mg, or 120-140 mg of etoricoxib per day to the subject. In some embodiments, the pharmaceutical composition may comprise 20-40 mg, 40-60 mg, 60-80 mg, 80-100 mg, 100-120 mg, or 120-140 mg of etoricoxib. In some embodiments, the method may comprise administering 30 mg, 40 mg, 50 mg, 60 mg, 70 mg, 80 mg, 90 mg, 100 mg, 110 mg, or 120 mg of etoricoxib. In some embodiments, the pharmaceutical composition may comprise 30 mg, 60 mg, 90 mg, or 120 mg of etoricoxib for oral or intravenous administration. In some embodiments, the method is more effective at treating the viral hemorrhagic fever and/or related symptoms than acetaminophen (paracetamol).

In one embodiment, a method is provided for treating viral hemorrhagic fever in a subject, wherein the method comprises administering an initial dose of etoricoxib to the subject, and thereafter administering a maintenance dose of etoricoxib to the subject. In one aspect, the initial dose may comprise 90 to 500 mg per day of etoricoxib. In another aspect, the method may comprise administering the initial dose to the subject during the febrile phase of the infection. In another aspect, the method may comprise administering a maintenance dose of 30 to 120 mg per day of etoricoxib to the subject after the completion of the initial dose. In a further aspect, the maintenance dose may be administered during the critical phase of the infection, the recovery phase of the infection, or both. In another aspect, the method may comprise treating one or more symptoms of the viral hemorrhagic fever, such as fever, pain, or inflammation. In a further aspect, the method may comprise treating the subject to reduce the viral level of the virus causing the viral hemorrhagic fever in the subject. In another aspect, the maintenance dose is administered until the subject experiences a reduction in at least one or more symptoms of the viral hemorrhagic fever. In a further aspect, the method comprises treating the subject through administration of a pharmaceutical composition comprising etoricoxib and a pharmaceutically acceptable carrier.

In certain aspects, a method is provided for treating a subject infected with the Zika virus, the method comprising administering to the subject etoricoxib. In some embodiments, the method comprises administering an amount of etoricoxib sufficient to cause a reduction in the viral level of the Zika virus. In some embodiments, the method comprises administering an amount of etoricoxib sufficient to cause reduction in the subject of one or more symptoms of the Zika virus.

Without limiting the scope of this disclosure, in any of the methods described herein, the treating of the viral hemorrhagic fever may comprise the treating of one or more symptoms of the viral hemorrhagic fever, including fever, pain, or inflammation; the viral hemorrhagic fever may be Dengue fever; the subject may be a human patient (adult or child); the pharmaceutical composition may be orally administered or suitable for oral administration, such as a solid dosage form (tablet or capsule); the method may comprise further administering acetaminophen to the subject; and the method may be performed without increasing the risk of a bleeding event or adverse cardiovascular event in the subject. The methods may further comprise providing to the subject a product comprising a quantity of etoricoxib sufficient for a single course of therapy. In some embodiments, the single course of therapy may be provided through a container comprising a pre-defined number of solid dosage forms of a pharmaceutical composition comprising etoricoxib and a pharmaceutically acceptable carrier, as described below.

In certain aspects, a product is provided for the short-term use of etoricoxib, the product comprising a container comprising a plurality of solid dosage forms of a pharmaceutical composition comprising etoricoxib and a pharmaceutically acceptable carrier. In some embodiments, the solid dosage form is a tablet or capsule. In some embodiments, the container is a blister pack. In some embodiments, the blister pack comprises a plurality of cavities configured to hold a solid dosage form of etoricoxib comprising a tablet or capsule. In some embodiments, the container comprises between 2 and 14 individual solid dosage form units of a pharmaceutical composition comprising etoricoxib and a pharmaceutically acceptable carrier. In some embodiments, at least two of the individual solid dosage form units comprise different amounts of etoricoxib. The product may comprise a set of instructions for dosing and use of the pharmaceutical composition comprising etoricoxib and a pharmaceutically acceptable carrier.

References for the Description

The following documents are incorporated by reference in their entirety:
Dallob, A., Hawkey, C. J., Greenberg, H., Wight, N., De Schepper, P., Waldman, S., Gottesdiener, K. (2003). Characterization of Etoricoxib, a Novel, Selective COX-2 Inhibitor. The Journal of Clinical Pharmacology, 43(6), 573-585.
European Medical Agency, Arcoxia 30 60 90 120 mg film-coated tablets—Summary of Product Characteristics (SPC)-(eMC), Last Updated on eMC 25 Jul. 2016
Australian Register of Therapeutic Goods, ARCOXIA Tablet PI (MK0663-AUS-2015-011356), Last Updated Jul. 14, 2017
Diaz-Quijano et al. Predictors of spontaneous bleeding in patient with acute febrile syndrome from a dengue endemic area J Clin Virology 49 (2010) 11-15
US Food and Drug Administration. VIOXX (Rofecoxib) U.S. Prescribing Information May 9, 2016
Epidemiology Unit, Ministry of Health, Sri Lanka. Advice for Dengue patients who are on home based care temporarily, 2017 (http://www.epid.gov.lk/web/images/pdf/DHF/Leaflet/dengue_leaflet_english.pdf)
United Nations, Caring-for-a-Dengue-patient, 2017 (http://lk.one.un.org/wp-content/uploads/2017/07/Caring-for-a-Dengue-patient.pdf)
World Health Organization, Dengue Fact Sheet, http://www.who.int/mediacentre/factsheets/fs117/en/
Sameh Elawdy, et al. Association between severity of dengue infection and dengue viral load: a systematic review and meta-analysis. PROSPERO 2016 CRD42016039864 http://www.crd.york.ac.uk/PROSPERO/display_record.php?ID=CRD42016039864
Coxib and traditional NSAID Trialists' (CNT) Collaboration. Vascular and upper gastrointestinal effects of non-steroidal anti-inflammatory drugs: meta-analyses of individual participant data from randomised trials. The Lancet 2013; 382: 769-79.
Cannon, C. P., Curtis, S. P., FitzGerald, G. A., Krum, H., Kaur, A., Bolognese, J. A., Laine, L. Cardiovascular outcomes with etoricoxib and diclofenac in patients with osteoarthritis and rheumatoid arthritis in the Multinational Etoricoxib and Diclofenac Arthritis Long-term (MEDAL) programme: a randomised comparison. The Lancet 2006, 368(9549), 1771-1781.
Riendeau, D., Percival, M. D., Brideau, C., Charleson, S., Dubé, D., Ethier, D., . . . Chan, C.-C. (2001). Etoricoxib (MK-0663): Preclinical Profile and Comparison with Other Agents That Selectively Inhibit Cyclooxygenase-2. Journal of Pharmacology and Experimental Therapeutics, 296(2), 558.
Laine L., Curtis S P, Cryer B, Kaur A, Cannon C P, for the MEDAL Steering Committee. Assessment of upper gastrointestinal safety of etoricoxib and diclofenac in patients with osteoarthritis and rheumatoid arthritis in the Multinational Etoricoxib and Diclofenac Arthritis Long-term (MEDAL) programme: a randomised comparison. The Lancet 2007; 369: 465-73.
McGettigan P, Henry D. Cardiovascular risk with non-steroidal anti-inflammatory drugs: systematic review of population-based controlled observational studies. PLoS Med 2011; 8: e1001098.
Malmstrom, K., Sapre, A., Couglin, H., Agrawal, N. G., Mazenko, R. S., & Fricke, J. R. Etoricoxib in acute pain associated with dental surgery: A randomized, double-blind, placebo- and active comparator-controlled dose-ranging study. Clinical Therapeutics 2004, 26(5), 667-679.
Daniels, S. E., Bandy, D. P., Christensen, S. E., Boice, J., Losada, M. C., Liu, H., Peloso, P. M. Evaluation of the dose range of etoricoxib in an acute pain setting using the postoperative dental pain model. The Clinical Journal of Pain 2011, 27(1), 1-8.
Rawal, N., Viscusi, E., et al. Evaluation of etoricoxib in patients undergoing total knee replacement surgery in a double-blind, randomized controlled trial. BMC Musculoskeletal Disorders 2013; London, 14, 300.
Viscusi, E. R., et al. Perioperative use of etoricoxib reduces pain and opioid side-effects after total abdominal hysterectomy: a double-blind, randomized, placebo-controlled phase III study. Current Medical Research and Opinion 2012, 28(8), 1323-1335.
Takemoto, J. K., et al. Clinical Pharmacokinetic and Pharmacodynamic Profile of Etoricoxib. Clinical Pharmacokinetics 2008, 47(11), 703-720.

### EXAMPLES

### Example 1—a Randomized, Open-Label, Active-Comparator-Controlled, Phase IV Pilot Study to Evaluate the Efficacy and Safety of Etoricoxib in Subjects with Dengue Fever; Study Protocol

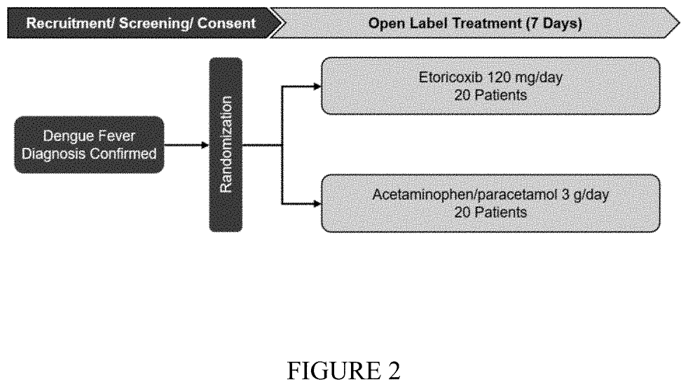

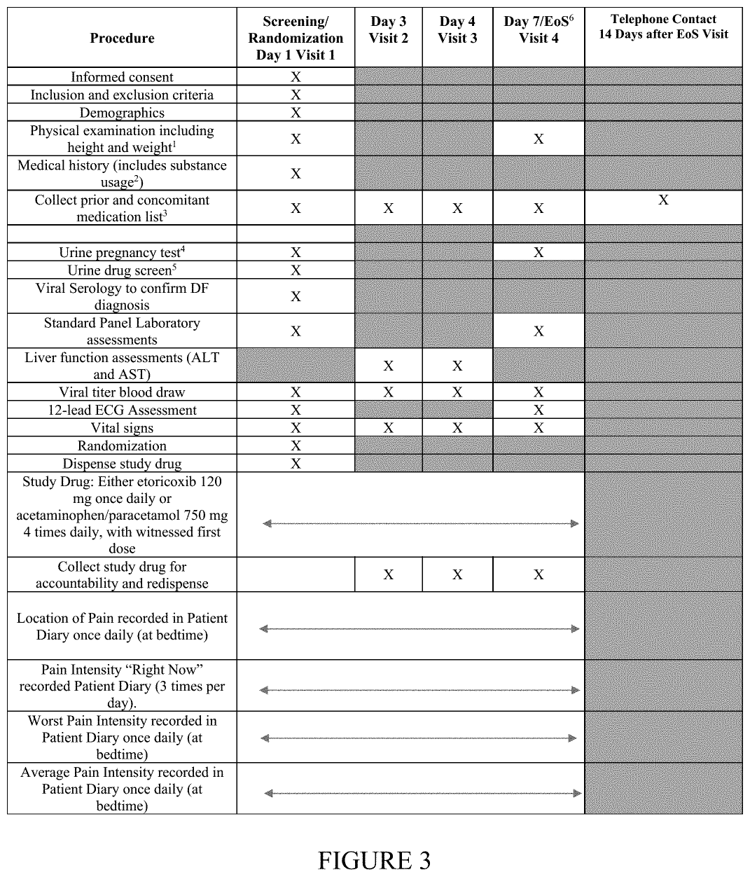

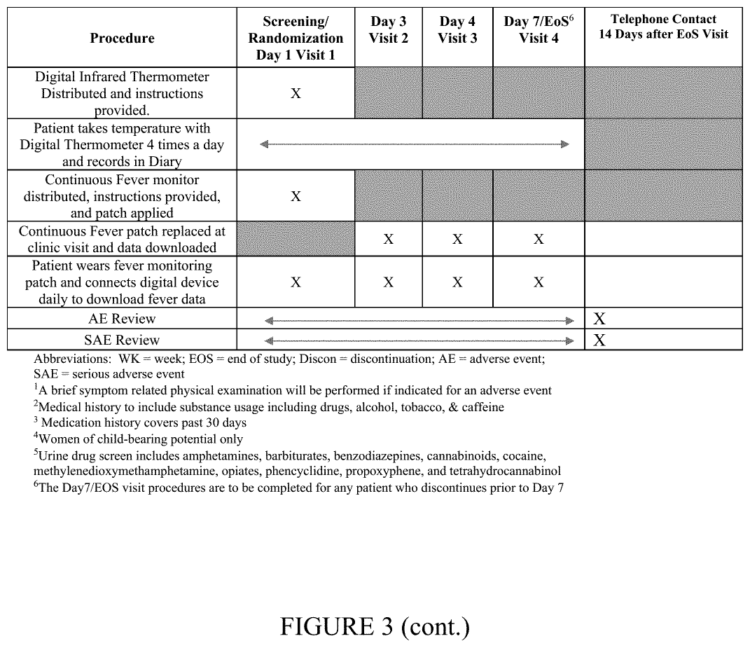

FIG. 2 shows an exemplary schematic of the study. In one embodiment, the study includes two main phases. In phase 1, subjects can be recruited, screened and the consent is obtained. Dengue fever diagnosis is confirmed and recruited subjects are randomized in treatment groups. Phase 2 can include a 7-day open-label treatment. An experimental group of about 20 subjects is administered 120 mg/day etoricoxib. A control group of 20 subjects is administered the current compare standard of care treatment of 3 g/day acetaminophen (paracetamol). FIG. 3 shows the schedule of subject assessments.

Introduction

Etoricoxib is a cyclooxygenase-2 (COX 2) selective, non-steroidal anti-inflammatory drug (NSAID) with proven analgesic, anti-inflammatory, and anti-pyretic properties {1} being developed to treat dengue fever (DF), an illness with symptoms including high fever and intense pain. COX-2 is highly expressed in dengue fever virus-infected cells {2}. COX-2 is the isoform of the enzyme that has been shown to be induced by pro-inflammatory stimuli and has been postulated to be primarily responsible for the synthesis of the prostanoid mediators of pain, inflammation and fever through its metabolite prostaglandin E2 (PGE2) {1}. Because etoricoxib is highly selective for the COX-2 isoenzyme of cyclooxygenase, it is expected that etoricoxib will suppress the synthesis of prostaglandins and reduce the pain, inflammation and fever associated with dengue fever.

There is no current treatment specifically approved to treat dengue fever {3,4,5}. Nonsteroidal anti-inflammatory drugs (NSAIDs) are currently contraindicated by WHO for the treatment of dengue fever, primarily due to concern about increased bleeding risk in conjunction with thrombocytopenia commonly associated with the illness {3}. Acetaminophen/paracetamol is the standard of care to reduce fever and pain {3}. However, acetaminophen/paracetamol is associated with an elevated risk of liver failure {6} and given that hepatitis is a frequent dengue complication {3,7}, it is reasonable to consider the potential for increased risk of liver injury with acetaminophen/paracetamol use in subjects with dengue fever. In one embodiment, the subject matter described relates to the surprising discovery that the clinical limitations of certain COX-2 selective NSAIDs are not applicable to subjects suffering from an acute, short duration (often less than 14 days), high morbidity, potentially high mortality, and self-limiting condition, such as, for example, a VHF, and that the short-term use of etoricoxib can provide significant benefits to subjects suffering from VHFs, including Dengue fever. In addition, as fever severity in subjects with VHFs is correlated with disease morbidity and possibly outcomes, it has been surprisingly discovered that direct pharmacological intervention with etoricoxib, due in part to its potent anti-pyretic effect, for a short duration can address fever in these subjects in such a way to influence the outcome of the disease, while avoiding the toxicities associated with long-term use of these agents. Furthermore, it has been surprisingly discovered that direct pharmacological intervention with etoricoxib, which is a potent COX-2 selective NSAID with a long half-life and sustained inhibitory effect for a short duration may suppress COX-2 replication in these subjects, reduce disease severity, and minimize or avoid the toxicities associated with long-term use of COX-2 selective NSAIDs. It has also been surprisingly discovered that etoricoxib may also be effective in treating a viral hemorrhagic fever, including associated fever, pain and inflammation, in subjects infected with a virus capable of causing a viral hemorrhagic fever, such as Dengue fever. It has also been surprisingly discovered that etoricoxib may be safely used to treat a viral hemorrhagic fever, including associated fever, pain and inflammation, in a subject without increasing the risk of bleeding or other adverse platelet effects; gastritis or other gastrointestinal-related side effects or adverse events; or cardiovascular-related side effects or adverse events in the subject.

Observed interactions between dengue infection and the COX-2 signaling cascade have demonstrated elevated COX-2 expression and elevated levels of its prostaglandin E2 (PGE2) {7}. Additional research on dengue-infected hepatoma cells demonstrated induction of COX-2 RNA and protein levels, along with increased PGE2 levels {2}. In addition, COX-2 overexpression and the addition of exogenous PGE2 enhanced DENV replication in a dose dependent fashion {2,8}. Conversely, COX-2 inhibition was found to inhibit DENV replication in hepatoma cells and in a suckling mouse model {2}. It has been discovered that targeting COX-2 via etoricoxib, a COX-2 selective inhibitor, may present an antiviral development opportunity {2,8}.

Study Rationale

Dengue Fever is a serious, flu-like illness that affects infants, young children and adults in the tropical and subtropical regions of the world, mostly in urban and semi-urban setting {5}. It is a mosquito-borne flavivirus spread by day-biting Aedes mosquitos. It is the fastest spreading vector-borne viral disease and is endemic in 128 countries, resulting in 3.9 billion people at risk globally {5}. It is caused by one of four distinct serotypes (dengue virus (DENV) -1 through -4). While the first infection with one of the four dengue serotypes is typically asymptomatic or non-severe, individuals who are subsequently exposed to one of the other serotypes are more likely to develop severe dengue. {3,10}

Dengue has a wide spectrum of clinical presentations, often with unpredictable clinical evolution and outcome. While most subjects recover following a self-limiting clinical course, a small proportion progress to severe disease characterized by plasma leakage with or without hemorrhage. The group progressing from non-severe to severe disease is difficult to identify but this is an important concern since appropriate treatment may prevent these subjects from developing more severe clinical conditions {3}.

Dengue infection occurs when the virus enters the skin along with the mosquito's saliva, infecting dendritic cells (DC), a form of white blood cell {9}. The virus uses the replication machinery of the host cell to translate its RNA into a polypeptide, beginning the cycle of viral replication. White blood cells create an immune response to the virus using signaling proteins, including interferons and other cytokines {9}.

Symptoms of dengue fever usually last for 2-7 days {3}. Symptoms of dengue fever include high fever and two or more of the following: severe headache, pain behind the eyes, muscle and joint pains, nausea, vomiting, swollen glands, and rash. Severe dengue, including dengue hemorrhagic fever or dengue shock syndrome, is characterized by severe abdominal pain, persistent vomiting, rapid breathing, bleeding gums, fatigue, restlessness, and blood in vomit, and may be fatal due to plasma leakage, fluid accumulation, respiratory distress, severe bleeding, or organ impairment {3}. Case fatality rates can be below 1% with proper case management. In its absence of proper management, the case fatality rate can be as high at 20% in subjects with severe dengue {10}.

There is no treatment specifically approved for dengue fever {3,4}. WHO and CDC guidelines recommend fluids, bedrest as well as acetaminophen/paracetamol and tepid baths to reduce fever and improve subject comfort {3,4}. WHO recommends that acetaminophen/paracetamol be used as an antipyretic {3}. The recommended dose of acetaminophen/paracetamol is up to 4 g daily, dosed at up to 1000 mg every 6 hours.

However, subjects with dengue fever are at risk for hepatic injury {11,12, 13}. Elevated concentrations of AST and ALT are associated with the disease {11, 12, 13, 14, 15} and dengue is a major source of liver failure in tropical countries in which dengue fever is prevalent {13} and the use of acetaminophen/paracetamol is associated with known liver toxicity.

Several studies have demonstrated that acetaminophen/paracetamol intake is associated with transaminase elevation in dengue subjects. A multicenter, randomized controlled trial further confirmed that even a modest daily dose of acetaminophen/paracetamol (median of 1.5 g) used as an antipyretic in dengue infection caused transaminase elevation {14}. This finding is consistent with case reports of fulminant hepatic failure, in which most subjects reported acetaminophen/paracetamol ingestion at a dose within the therapeutic range recommended by WHO, and the US Food and Drug Administration {15,16}. This study also found that acetaminophen/paracetamol intake did not appear to effect mean or maximum body temperature. In addition, pain scores, analgesic intake, length of stay, or duration of fever for the acetaminophen/paracetamol group was not significantly different from placebo, calling into question the efficacy as well as the safety of acetaminophen/paracetamol in dengue fever. Two observational studies reported an association between excessive acetaminophen/paracetamol intake and transaminase elevation in dengue subjects {12,3}. A retrospective study {12} found that an acetaminophen/paracetamol dose of more than 60 mg/kg/day before admission was significantly associated with the occurrence of hepatitis. This study concluded that acetaminophen/paracetamol doses should not exceed 3 g/day, less than that recommended by WHO. Another retrospective study {13} reported that one subgroup of subjects reporting previous acetaminophen/paracetamol intake within 24 h was significantly associated with transaminase elevation.

It has been surprisingly discovered that etoricoxib may be used to treat dengue fever, including associated symptoms such as fever, pain, and inflammation, without the risk of liver injury associated with acetaminophen/paracetamol. It has also been surprisingly discovered that etoricoxib may be safely used to treat a viral hemorrhagic fever, including associated fever, pain and inflammation, in a subject without increasing the risk of bleeding or other adverse platelet effects; gastritis or other gastrointestinal-related side effects or adverse events; or cardiovascular-related side effects or adverse events in the subject. For these reasons, etoricoxib may provide a more efficacious alternative with less risk relative to acetaminophen/paracetamol in treating the pain and fever in subjects with dengue fever.

COX-2 is highly expressed in DENV-infected cells {2,7,8}. The COX-2 enzyme directly regulates the catalysis of arachidonic acid to prostaglandins (PGs). PGs play a role in development of immune responses by stimulating inflammatory cell chemotaxis, amplifying the formation of pain impulses, causing vasodilation, and signaling the hypothalamus to increase the body's temperature in response to pyrogens. Highly selective COX-2 inhibitors selectively block the activity of the COX-2 enzyme, effectively decreasing PG production, in turn decreasing pain, inflammation and fever {17, 2, 8}.

Etoricoxib is a highly selective COX-2 inhibitor with demonstrated efficacy in reducing pain {1}. Etoricoxib is approved in over 80 countries for the treatment of OA, RA, acute gout, and acute pain associated with surgery, primary dysmenorrhea and for chronic musculoskeletal pain. The countries in which etoricoxib is approved include Guatemala {18} and many others where dengue fever is prevalent, such as Brazil, Honduras, Singapore, Malaysia, Thailand, India and Pakistan.

The antipyretic effect of etoricoxib has also been demonstrated. It has been observed that the COX-2 isoform is primarily involved in the genesis of fever in humans. A study of inhibition of COX-2 by another highly selective COX-2 inhibitor, rofecoxib, resulted in antipyretic activity in monkeys and humans, similar to the non-selective COX-1/COX-2 inhibitors, diclofenac and ibuprofen {19}. This research supports the hypothesis that the inhibition of COX-2 can reduce fever. In clinical trials of the use of etoricoxib to treat acute pain after surgery, subjects treated with etoricoxib had fewer fever AEs than subjects on placebo {20,21, 22}. Consistent with this observation, the etoricoxib label carries a warning that it may mask fever {1}.

Etoricoxib is not associated with bleeding adverse events {1}. The administration of therapeutic doses of etoricoxib in healthy subjects does not affect COX-1 activity in circulating platelets {23}. Ex vivo whole-blood assays after multiple oral doses of etoricoxib showed no important effects on bleeding time or platelet aggregation {25}. In acute pain studies, etoricoxib was not associated with an increased risk of bleeding {27,28, 29, 30, 31}.

A clinical study of etoricoxib conducted in subjects with hemophilia also provides evidence of the lack of an effect on bleeding in a population of subjects at high risk of bleeds {31}. In this double-blind, placebo-controlled pilot study, subjects with hemophilic arthropathy, an arthritic condition caused by repeated joint bleeds, demonstrated that treatment with etoricoxib was not associated with an increase in joint bleeding. In Part 1 of the study (6 weeks), 102 subjects with hemophilic arthropathy were randomized to receive either etoricoxib 90 mg or placebo. In Part 2, 51 subjects taking placebo in Part 1 were randomized to receive either etoricoxib 90 mg or rofecoxib 25 mg for 6 months. Subjects in Part 1 continued to take etoricoxib. During the 6-week portion of the study the incidence of joint bleeding was generally similar between the etoricoxib and the placebo groups (66.7% for etoricoxib and 72.6% for placebo), indicative that etoricoxib does not further exacerbate bleeding. During the 6-month extension, the incidence of joint bleeding was also generally similar between the etoricoxib and placebo groups (78.9% for etoricoxib and 77.0% for placebo). There were two serious AEs (3.9%) during the placebo-controlled portion of the study, both in subjects on etoricoxib. One had traumatic arthropathy and hemarthrosis and another subject had a bleeding duodenal ulcer. The subject with the bleeding duodenal ulcer discontinued the study. In addition to the lack of bleeding risk, the use of etoricoxib is not associated with the magnitude of liver function abnormalities associated with acetaminophen/paracetamol. The chronic use of etoricoxib (for up to one year in clinical trials) has been associated with elevated of ALT or AST (≥3 times the upper limit of normal) in 1% of subjects, an observation consistent with the use of other NSAIDs not specifically associated with liver injury.

In addition to its role in treating the inflammation, pain and fever associated with dengue fever, there is also evidence that etoricoxib may suppress the replication of the DENV {7, 2, 8}. COX-2 is not only an important mediator of inflammation in response to viral infection, but it has also been demonstrated to play a role in viral replication in other illnesses, for instance in Hepatitis C replication. An increased level of COX-2 has been seen in subjects with dengue fever compared to healthy donors, as well as in DENV-infected ICR suckling mice {2}.

In-vitro experiments have found that the DENV infection significantly induced COX-2 expression and PGE2 in human hepatoma cells, and that COX-2 gene silencing and catalytic inhibition suppressed DENV-replication {2}. In a suckling mouse model, it was also demonstrated that COX-2 inhibition protected mice from succumbing to the DENV-2 infection {2}. These studies demonstrated that COX-2 is an important factor in DENV replication and may serve as a target for anti-viral drug development {2,8}. As a highly selective COX-2 inhibitor etoricoxib may have a beneficial effect in suppressing DENV-replication.

Etoricoxib is being studied for the treatment of dengue fever based on its mechanism of action, its demonstrated efficacy in treating acute pain and fever, its lack of effect on platelet function and its corresponding lack of effect on bleeding in acute situations, the expected safety advantage on liver function relative to the standard of care (acetaminophen/paracetamol), and its potential benefit in suppressing DENV-replication. This pilot study is intended to provide evidence of the benefit of the efficacy and safety of etoricoxib versus the standard of care, acetaminophen/paracetamol.

Benefit Risk Assessment

There is no treatment specifically approved for the signs and symptoms of dengue fever {3,4}. There is a high unmet need for an agent to manage the symptoms associated with dengue fever that may potentially be met with etoricoxib. In one embodiment, the subject matter described relates to the surprising discovery that the clinical limitations of certain COX-2 selective NSAIDs are not applicable to subjects suffering from an acute, short duration (often less than 14 days), high morbidity, potentially high mortality, and self-limiting condition, such as, for example, a VHF, and that the short-term use of etoricoxib at the dose of 120 mg/day can provide significant benefits to subjects suffering from VHFs, including Dengue fever. In addition, as fever severity in subjects with VHFs is correlated with disease morbidity and possibly outcomes, it has been surprisingly discovered that direct pharmacological intervention with etoricoxib, due in part to its potent anti-pyretic effect, for a short duration can address fever in these subjects in such a way to influence the outcome of the disease, while avoiding the toxicities associated with long-term use of these agents. Furthermore, it has been surprisingly discovered that direct pharmacological intervention with etoricoxib, a potent COX-2 selective NSAID with a long half-life and sustained inhibitory effect for a short duration may suppress COX-2 replication in these subjects, reduce disease severity, and minimize or avoid the toxicities associated with long-term use of COX-2 selective NSAIDs. It has been surprisingly discovered that etoricoxib at the dosage of 120 mg/day may also be effective in treating a viral hemorrhagic fever, including associated fever, pain and inflammation, in subjects infected with a virus capable of causing a viral hemorrhagic fever, such as Dengue fever. It has also been surprisingly discovered that etoricoxib may be safely used to treat a viral hemorrhagic fever, including associated fever, pain and inflammation, in a subject without increasing the risk of bleeding or other adverse platelet effects; gastritis or other gastrointestinal-related side effects or adverse events; or cardiovascular-related side effects or adverse events in the subject

It is critical that any treatment for dengue fever not be associated with bleeding risk. As a highly-selective COX-2 inhibitor, etoricoxib does not impact platelet function or associated bleeding risk in acute situations {1}.

The unmet need is particularly high in dengue fever because the standard of care is associated with increased risk of liver injury {32,33}, and dengue fever is a disease state in which subjects are at risk of hepatic toxicity {34}. Etoricoxib fulfills this long-felt but unmet need by providing a liver function safety advantage relative to the standard of care, acetaminophen/paracetamol.

Acetaminophen/paracetamol is a widely used nonprescription analgesic and antipyretic medication for mild-to-moderate pain and fever {32}. While acetaminophen/paracetamol is considered to be associated with low safety risk at low doses, it has direct hepatotoxic potential when taken at higher than recommended doses and can cause acute liver injury and death from acute liver failure {6, 32}. Even at recommended doses, acetaminophen/paracetamol can cause transient serum aminotransferase elevations. Chronic therapy with acetaminophen/paracetamol in doses of 4 grams daily has been found to lead to transient elevations in serum aminotransferase levels in a proportion of subjects, generally starting after 3 to 7 days, and with peak values of ≥3 times the upper normal limit in 39% of persons {6}. Severe acetaminophen/paracetamol hepatoxicity frequently leads to acute liver failure {33}. In 2005 Larson reported that acetaminophen/paracetamol accounted for 42% of all cases of acute liver failure in academic centers participating in the Acute Liver Failure Study Group. {33}. Using a population-based approach covering metropolitan Atlanta, Ga., Bower reported in 2007 that 41% of all cases of acute liver failure were secondary to acetaminophen/paracetamol {35}. In the United States, cases of acetaminophen/paracetamol acute liver failure are evenly distributed between suicide attempts and therapeutic uses {37}.

The mechanism of acetaminophen/paracetamol hepatotoxicity has been extensively analyzed in humans and in animal models {32,33}. Acetaminophen/paracetamol is largely converted to nontoxic glucuronate or sulfate conjugates and secreted in the urine. A minor amount of acetaminophen/paracetamol is metabolized via the cytochrome P450 system to intermediates that can be toxic. Ordinarily, this intermediate is rapidly conjugated to reduced glutathione, detoxified and secreted. If levels of glutathione are low or the pathway is overwhelmed by high doses of acetaminophen/paracetamol, the reactive intermediate accumulates and binds to intracellular macromolecules that can lead to cell injury, usually through apoptotic pathways. Factors that increase the metabolism of acetaminophen/paracetamol through the P450 system (certain drugs, chronic alcohol use) or that decrease the availability of glutathione (fasting, malnutrition, alcoholism) can predispose to acetaminophen/paracetamol toxicity.

Hepatic dysfunction is also well reported feature in both dengue fever and dengue hemorrhagic fever {34}. Liver involvement in dengue infection can be varied, ranging from mild to moderate elevation of serum transaminases to fulminant liver failure [12-14}. Various mechanisms have been suggested to explain the hepatic dysfunction seen in dengue, including direct viral damage, immunological injury and hypoxic injury due to hepatic perfusion during shock {12-14}. Use of acetaminophen/paracetamol carries significant risk of hepatic injury in subjects with dengue Fever who are more susceptible to its adverse hepatic effects.

Studies confirm that acetaminophen/paracetamol is associated with risk of liver injury in dengue fever subjects. A multicenter, randomized controlled trial further confirmed that even a median daily dose of 1.5 g of acetaminophen/paracetamol used as an antipyretic in dengue infection caused transaminase elevation {14}1. At a median daily dose of 1.5 g, the study also found that acetaminophen/paracetamol did not impact mean or maximum fever or separate from placebo on pain scores, calling into question the efficacy as well as the safety of acetaminophen/paracetamol for dengue fever. A retrospective study showed transaminase elevation at a dose of more than 60 mg/kg/day {12,13} The body of evidence suggests that an alternative treatment is required for the symptoms of dengue fever that has equal to or better efficacy than acetaminophen/paracetamol on pain and fever but has less risk of hepatic injury while not raising bleeding risk.

It has been surprisingly discovered that etoricoxib can provide the benefits of better efficacy and safety in the treatment of dengue fever versus acetaminophen/paracetamol. In one embodiment, the subject matter described relates to the surprising discovery that the clinical limitations of certain COX-2 selective NSAIDs are not applicable to subjects suffering from an acute, short duration (often less than 14 days), high morbidity, potentially high mortality, and self-limiting condition, such as, for example, a VHF, and that the short-term use of etoricoxib can provide significant benefits to subjects suffering from VHFs, including Dengue fever. In addition, as fever severity in subjects with VHFs is correlated with disease morbidity and possibly outcomes, it has been surprisingly discovered that direct pharmacological intervention with etoricoxib, due in part to its potent anti-pyretic effect, for a short duration can address fever in these subjects in such a way to influence the outcome of the disease, while avoiding the toxicities associated with long-term use of these agents. Furthermore, it has been surprisingly discovered that direct pharmacological intervention with etoricoxib, a potent COX-2 selective NSAID with a long half-life and sustained inhibitory effect for a short duration may suppress COX-2 replication in these subjects, reduce disease severity, and minimize or avoid the toxicities associated with long-term use of COX-2 selective NSAIDs. It has been surprisingly discovered that etoricoxib may also be effective in treating a viral hemorrhagic fever, including associated fever, pain and inflammation, in subjects infected with a virus capable of causing a viral hemorrhagic fever, such as Dengue fever. It has also been surprisingly discovered that etoricoxib may be safely used to treat a viral hemorrhagic fever, including associated fever, pain and inflammation, in a subject without increasing the risk of bleeding or other adverse platelet effects; gastritis or other gastrointestinal-related side effects or adverse events; or cardiovascular-related side effects or adverse events in the subject.

Data suggests that all NSAIDs, including etoricoxib (with the exception of high dose naproxen) appear to be associated with an increased risk of thrombotic CV events. The CV safety for etoricoxib was formally reviewed by the European Union with the conclusion that the benefit/risk remained positive {36}. Of note, the assessment of the risk of thrombotic CV events associated with the use of NSAIDs was most clearly focused on chronic use of NSAIDs rather than the short-course (7 days) to be evaluated in the current study.

In one embodiment, the subject matter described relates to the surprising discovery that the clinical limitations of certain COX-2 selective NSAIDs are not applicable to subjects suffering from an acute, short duration (often less than 14 days), high morbidity, potentially high mortality, and self-limiting condition, such as, for example, a VHF, and that the short-term use of etoricoxib can provide significant benefits to subjects suffering from VHFs, including Dengue fever. In addition, as fever severity in subjects with VHFs is correlated with disease morbidity and possibly outcomes, it has been surprisingly discovered that direct pharmacological intervention with etoricoxib, due in part to its potent anti-pyretic effect, for a short duration can address fever in these subjects in such a way to influence the outcome of the disease, while avoiding the toxicities associated with long-term use of these agents. Furthermore, it has been surprisingly discovered that direct pharmacological intervention with etoricoxib, a potent COX-2 selective NSAID with a long half-life and sustained inhibitory effect for a short duration may suppress COX-2 replication in these subjects, reduce disease severity, and minimize or avoid the toxicities associated with long-term use of COX-2 selective NSAIDs. It has been surprisingly discovered that etoricoxib may also be effective in treating a viral hemorrhagic fever, including associated fever, pain and inflammation, in subjects infected with a virus capable of causing a viral hemorrhagic fever, such as Dengue fever. It has also been surprisingly discovered that etoricoxib may be safely used to treat a viral hemorrhagic fever, including associated fever, pain and inflammation, in a subject without increasing the risk of bleeding or other adverse platelet effects; gastritis or other gastrointestinal-related side effects or adverse events; or cardiovascular-related side effects or adverse events in the subject. As a highly selective COX-2 inhibitor, etoricoxib does not present increased bleeding risk, which is critical for subjects with dengue fever who are susceptible to hemorrhage.

In vitro studies and a study in an animal model have found that COX-2 inhibition can suppress DENV-replication {2,8}. These studies have concluded that COX-2 is a potential target for developing therapeutic agents against the DENV infection. As a highly selective COX-2 inhibitor, etoricoxib may have an additional benefit in suppressing the replication of the DENY.

Therefore, with a clear need for an analgesic and anti-inflammatory medication with reduced risk of liver injury compared to the standard of care or of hemorrhagic complications, as well as a need for therapeutic agents to suppress viral replication, dengue fever represents a distinct disease state with a positive benefit-risk profile for the use of etoricoxib.

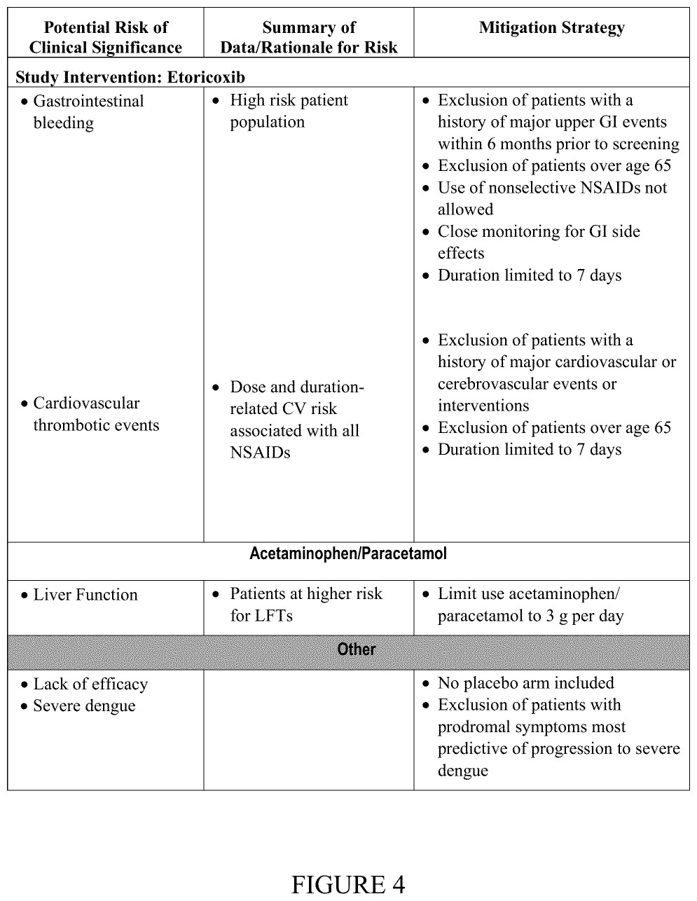

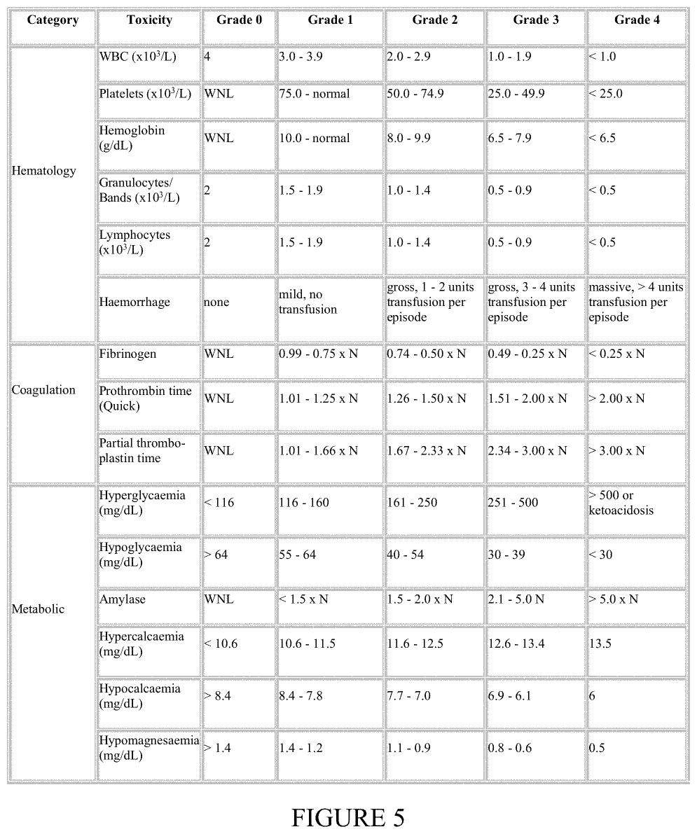

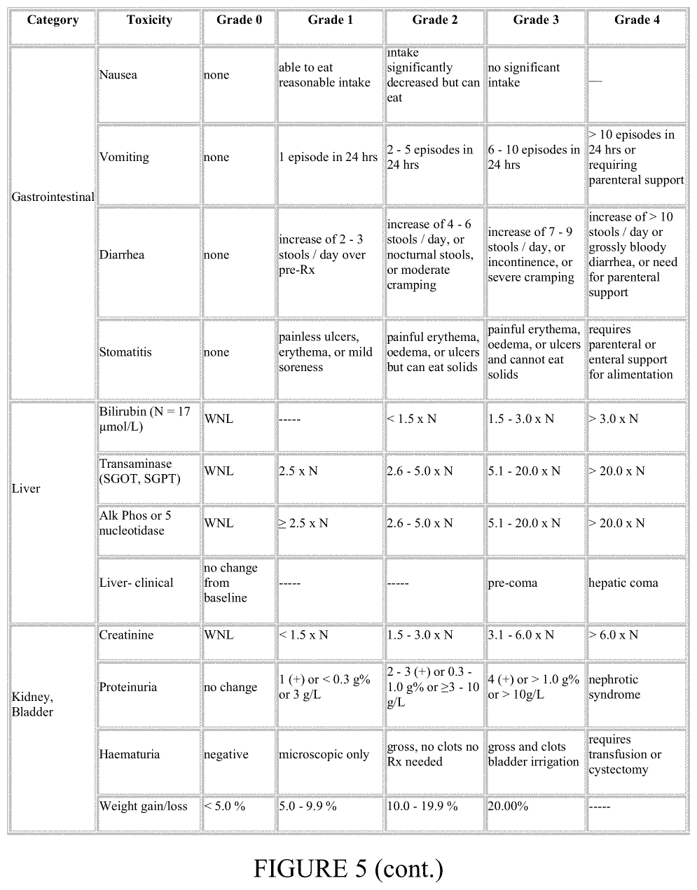

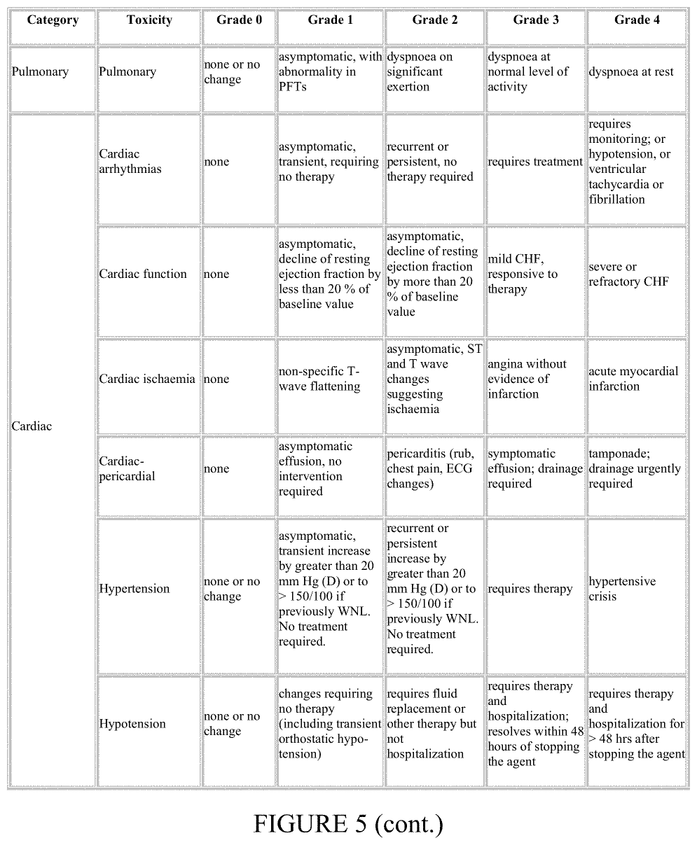

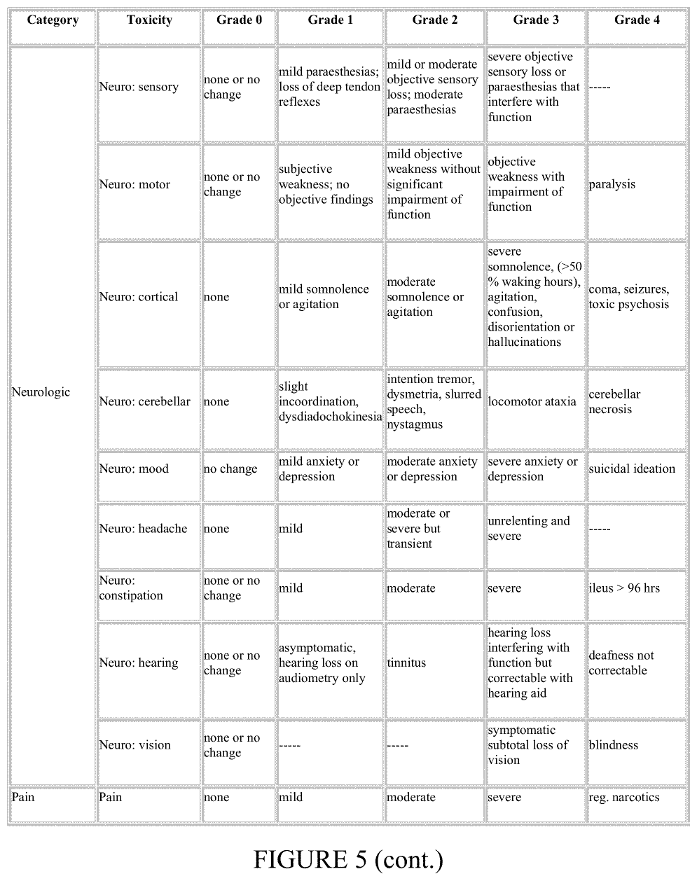

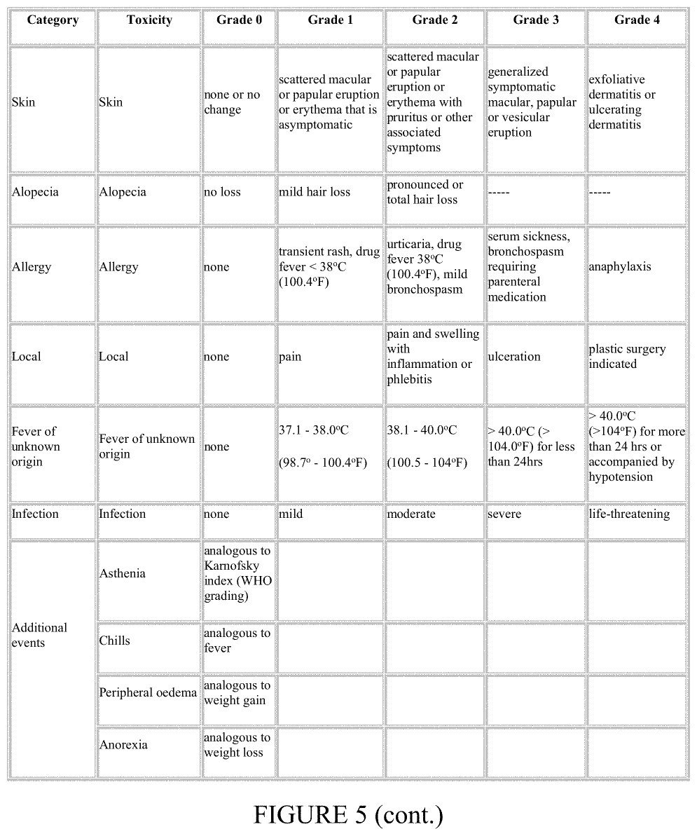

FIG. 4 shows minimization of risk strategy. To ensure standardization, all adverse events will be assessed using WHO Toxicity Criteria as shown in FIG. 5.

Dose Rationale

The 120 mg dose of etoricoxib was chosen for the clinical study in DF because this dose is approved in Guatemala for acute pain and is considered an appropriate dose to be studied in this condition which is also considered an acutely painful condition.

Trial Objectives, Endpoints and Purpose

Trial Objectives

Primary Objective:

The primary objective of the study is to evaluate the efficacy of etoricoxib 120 mg versus acetaminophen/paracetamol 3 g daily in subjects with dengue fever as measured by subject assessment of pain intensity (pain right now).

Key Secondary Objective:

The key secondary objective of the study is to evaluate the efficacy of etoricoxib 120 mg versus acetaminophen/paracetamol 3 g daily in reducing fever in subjects with dengue fever as measured by continuous and digital thermometer measurements.

Other Secondary Objectives:

Other secondary objectives include (1) evaluating the efficacy of etoricoxib 120 mg versus acetaminophen/paracetamol 3 g daily in subjects with dengue fever as measured by subject assessment of worst pain intensity; (2) evaluating the efficacy of etoricoxib 120 mg versus acetaminophen/paracetamol 3 g daily in subjects with dengue fever as measured by subject assessment of average pain intensity; (3) evaluating the safety of etoricoxib 120 mg versus acetaminophen/paracetamol 3 g daily in subjects with dengue fever based on liver function as measured by aspartate aminotransferase (AST) and alanine aminotransferase (ALT).

Exploratory Objective.

Exploratory objectives include (1) exploring the effect of etoricoxib 120 mg daily on viral load in subjects with dengue fever; (2) exploring the time course of pain relief for etoricoxib 120 mg versus acetaminophen/paracetamol 3 g daily in subjects with dengue fever based on subject assessment of pain right now and worst pain intensity.

Safety Objectives.

Safety objectives include evaluating the safety and tolerability of etoricoxib 120 mg daily in subjects with dengue fever based on adverse experiences, physical examinations, vital signs, clinical laboratory evaluations and electrocardiograms (ECGs).

Study Endpoints

Primary Endpoint:

The change from baseline in the subject assessment of how much pain they have right now on a 0- to 10-point Numeric Rating Scale (NRS) measured three time daily and averaged over the first 48 hours of treatment.

Key Secondary Endpoints:

The change from baseline in the time-weighted average (area under the curve divided by time interval) temperature over the first 48 hours of treatment as measured by continuous fever monitor readings and digital thermometer readings 4 times per day.

Other Secondary Efficacy Endpoints:

Secondary efficacy endpoints include (1) the change from baseline in the daily worst pain intensity scores on a 0- to 10-point NRS measured at bedtime and averaged over the first 48 hours of treatment; (2) the change from baseline in the daily average pain intensity score on a 0- to 10-point NRS measured at bedtime and averaged over the first 48 hours of treatment; (3) the change from baseline in aspartate aminotransferase (AST) and alanine aminotransferase (ALT) at 72 hours and at the end of the study (Day 7).

Exploratory Endpoint:

Exploratory endpoints include (1) the time weighted average difference from baseline in viral load (area under the log 10 transformed plasma viremia curve from day 1 to end of study (Day 7) divided by the time interval); (2) subject assessment of pain they have right now on a 0- to 10-point numeric rating scale (NRS) measured three times daily over the 7-day treatment period and subject assessment of worst pain intensity on a 0- to 10-point numeric rating scale (NRS) measured at bedtime over the 7-day treatment period.

Overall Study Design and Plan: Description

This is a randomized, open-label, active-comparator-controlled, parallel-group Phase IV Pilot study. The study is planned to evaluate the efficacy, safety, and tolerability of 120 mg etoricoxib and 3 g acetaminophen/paracetamol daily during dengue fever illness (7 days).

Subjects will be screened to determine eligibility and subjects who meet all of the inclusion criteria and none of the exclusion criteria will be randomized in a 1:1 ratio to either the etoricoxib 120-mg once daily arm or acetaminophen/paracetamol-750 mg 4 times daily (3 g) arm for 7 days. All subjects are to wear a continuous fever monitor and maintain a pain intensity diary. Subjects are also to take their temperature 4 times per day with a digital infrared thermometer. Subjects are to attend 4 clinic visits on study Day 1 (Visit 1/Randomization), Day 3 (Visit 2), Day 4 (Visit 3) and Day 7/End of Study (Visit 4) The total time on study is 7 days. The anticipated total number of clinic visits is 4.

This is an outpatient study. Subjects are to take their first dose of study medication in the clinic, but all subsequent doses will be taken on an outpatient basis. Doses of 120 mg etoricoxib are to be taken in the morning at approximately the same time each day regardless of the time the initial dose is taken on study Day 1. The first dose of 750 mg (375 mg×2) acetaminophen/paracetamol is to be taken in the morning at approximately the same time each day, and subsequent doses are to be taken every 6 hours for a total of 4 times (a total of 3 g). Subjects are to wear the continuous fever monitor patch and take their temperature 4 times per day with the digital infrared thermometer. Subjects are to complete the Subject Diary daily.

At each visit subjects will be assessed for safety and overall study compliance, including compliance with the Subject Diary. Vital signs, including temperature, will be taken at each clinic visit. The fever monitor patch will be replaced at each clinic visit and fever data will be downloaded at each visit. Blood and urine samples for full laboratory tests and viral titer will be taken on Visit 1/Day 1/Screening and Visit 4/Day 7/EOS. Blood draws for additional assessments of AST, ALT and viral titer will be taken at Visit 2/Day 3 and Visit 3/Day 4. If a clinic visit is not possible delegated site personnel will visit the subject at home.

Schedule of Assessments

For each subject, 4 scheduled visits including the screening visit will be performed. The overall Schedule of Assessments for the study is provided in FIG. 2. Visits will be at approximately the same time of day each time.

If the subject cannot attend a scheduled clinic visit, delegated site personnel must be scheduled to make a home visit to collect laboratory samples and perform any procedures, collect the samples for the laboratory tests and deliver the next Subject Diary. At each visit the subject will be asked to confirm that they can attend their next clinic visit. If they cannot attend, delegated site personnel will be scheduled for the home visit at approximately the same time as the originally scheduled visit. Note: the Day7/EOS/Visit 4 procedures are to be completed for any subject who discontinues prior to Day 7.

Assessments by Study Visit

Screening/Randomization Visit/Visit 1/Day 1

On day one the purpose of the study will be explained to prospective participants and written informed consent will be obtained as well as agreement for the use of subject information as required by local regulations. Inclusion/exclusion criteria will be reviewed. Medical history will be documented demographic information will be collected. Prior and concomitant medications that the subject is taking will be recorded. A complete physical examination will be performed including height, weight, and vital signs. Vital signs will be collected after the subject has been in a seated position for at least 5 minutes. A 12-lead ECG will be performed.

Blood and urine samples will be collected for clinical laboratory tests (viral serology) hematology, chemistry, and urinalysis as well as urine drug screen (UDS) and urine pregnancy test for women of child-bearing potential (WOCBP). A blood sample for the dengue fever viral titer test will be collected and prepared/stored as instructed in the laboratory manual in order to send to a biomolecular laboratory. Subjects will be randomized.

Study requirements will be reviewed, including instructions on dosing, completing the daily Subject Diary, using the digital infrared thermometer and wearing the continuous fever monitoring patch. Subjects will be counselled to call the clinic if they experience any unusual symptom on a day without a clinic visit. The assigned study drug will be dispensed.

The subject will be observed to take the first dose of the study drug and record the first dose in the Subject Diary. The subject will be given the Day 1, Day 2 and Day 3 Subject Diary. Subject ID number and the date will be written in. If the subject is in the etoricoxib group, the study staff will instruct the subject to cross out the extra three lines provided for recording the time they take their study medicine each day. If necessary, the subject will be instructed to shave under their arm. The electronic fever monitor patch will be applied and access to the electronic App for the fever monitor provided.

The timing of Visit 2/Day 3 will be confirmed. Visits will be at approximately the same time of day each time. If the subject cannot attend Visit 2/Day 3, schedule delegated site personnel to make a home visit. If a home visit is planned for Visit 2/Day 3, the delegated site personnel must also bring the Day 4 Subject Diary to the home visit. Then the subject will be discharged.

Visit 2/Day 3

Visit 2 is to be scheduled 2 days after Visit 1. Visits will be at approximately the same time of day each time. The study drug will be collected and its accountability will be confirmed confirm. Dosing compliance, digital thermometer compliance, and fever monitor patch will be reviewed. Completed Subject Diary cards will be collected and reviewed for completeness. Subjects will be retrained as necessary on dosing compliance, fever monitoring, and on diary completion. Subjects will be assessed for AEs and use of concomitant medications. A brief symptom-related physical examination will be performed if indicated by an AE.

Vital signs (pulse, body temperature, blood pressure, respiratory rates) will be obtained after subject has been in a seated position for 5 minutes. Blood samples will be collected for evaluation of AST and ALT by the local laboratory. Blood sample will be collected for the viral titer laboratory test and prepared/stored as instructed in the laboratory manual in order to send to a biomolecular laboratory.

Study drug will be dispensed and subjects will be reminded of study requirements including dosing instructions for study drug, use of the Subject Diary, the digital thermometer and fever monitoring patch use. The subject will be given the Diary for Day 4. The Subject ID number, the study day and the date will be recorded. If the subject is in the etoricoxib group, the study staff will instruct the subject to cross out the extra three lines provided for recording the time they take their study medicine each day. The data will be downloaded from the fever monitor patch and a new patch will be assigned and applied.

The timing of next visit will be confirmed. If the subject cannot attend Visit 3/Day 4, the delegated site personnel will be scheduled to make a home visit. If a home visit is planned for Visit 3/Day 4, the delegated site personnel will also bring the Day 5-7 Subject Diary to the home visit. The subject will be discharged from the visit.

Visit 3/Day 4

Visit 3/Day 4 will be scheduled one day after Visit 2/Day 3. Visits will be at approximately the same time of day each time. Study drug will be collected, study drug accountability will be confirmed and dosing compliance, digital thermometer compliance, and fever monitor patch will be reviewed. Completed Subject Diary cards will be collected and reviewed for completeness. Subjects will be assessed for AEs and use of concomitant medications. A brief symptom-related physical examination will be performed if indicated by an AE.

Vital signs (pulse, body temperature, blood pressure, respiratory rates) will be obtained after the subject has been in a seated position for 5 minutes. Blood samples will be collected for evaluation of AST and ALT by the local laboratory. A blood sample will be collected for the viral titer test. The sample will be prepared as instructed in the laboratory manual in order to send to a biomolecular laboratory.

Study drug will be dispensed and subjects will be reminded of study requirements including dosing instruction for study drug, use of the Subject Diary and use of the continuous fever monitor. The subject will be given the Diary for Days 5-7. The subject ID number, the study day, and the date will be filled in. If the subject is in the etoricoxib group, the study staff will instruct the subject to cross out the extra three lines provided for recording the time they take their study medicine each day. The data from the patch will be downloaded. A new patch will be assigned and applied. The nest visit will be confirmed. Visit 4/Day 7 EOS visit will be in the clinic. The subject will then be discharged from the visit.

Visit 4/Day 7/End of Study

Visit 4 will be scheduled 3 days after Visit 3/Day 4. Visit 4 will be at the clinic. The visit will be at approximately the same time of day as previous visits. The study drug will be collected, study drug accountability will be confirmed and dosing compliance, digital thermometer compliance, and fever monitor patch will be reviewed. Completed Subject Diary cards will be collected and reviewed for completeness. Subjects will be assessed for AEs and use of concomitant medications. Brief symptom-related physical examination will be performed if indicated by an AE.

Vital signs (pulse, body temperature, blood pressure, respiratory rates) will be obtained after the subject has been in a seated position for 5 minutes. A 12-lead ECG will be performed. Blood and urine samples will be collected for clinical laboratory tests hematology, chemistry, and urinalysis). The laboratory tests will include a urine pregnancy test for WOCBP. Blood sample will be collected for the viral titer test. Sample will be prepared as instructed in the laboratory manual in order to send to a biomolecular laboratory. The data will be downloaded from the fever monitor patch and the patch will be removed. At this point the subject's participation in the study will be completed except for the telephone contact noted below.

Telephone Contact

A member of the study staff will contact the subject 14 days after the EoS visit to assess for AEs and will also document any concomitant medication taken since the EoS visit.

Early Discontinuation Visit

Any subject who discontinues early is to have the End of Study (EoS) Visit assessments performed as noted for Visit 4 above. The Visit 4/Day 7/EOS visit must occur in the clinic including a Visit 4/Day 7 visit that occurs for a subject who is discontinuing early.

Inclusion Criteria

Subjects may be included in the study only if all the following criteria are met:
1. Is able and willing to give written informed consent (IC) and authorize the use of personal health information in order to protect subject confidentiality as required by local regulations.
2. Is a male or female subject aged 18 to 65 years inclusive at the time of signing consent.
3. Has elevated temperature of at least 38° Celsius for no more than 48 hours prior to screening.
4. Has rating based on how much pain you have right now at screening of ≥4 on a 0- to 10-point NRS.
5. Has at least two dengue fever pain symptoms: severe headache, pain behind the eyes, joint pain, muscle pain, bone pain.
6. Dengue fever is the primary source of pain.
7. Has Positive serological test for dengue fever and it has been confirmed that the symptoms are indicative of dengue fever.
8. Is able and willing to wear a temperature monitoring device and to use an electronic device to download data from the monitoring device.
9. Is able and willing to use the digital thermometer to record temperature 4 times per day.
10. Is able and willing to complete a diary to record assessment of pain and dosing with study drug.

Exclusion Criteria

Subjects are excluded from the study if they meet any of the following exclusion criteria:
1. Has hypotension, dehydration or petechia.
2. Has elevated LFTs (AST and ALT) ≥3 times the upper normal limit
3. Has an estimated creatine clearance (using Cockroft-Gault equation) of less than 30 ml/min.
4. Has a history of liver/renal disease.
5. Has asthma, urticaria or allergic-type reactions after taking aspirin or another NSAID.
6. Has a history of alcohol or substance abuse, that makes the subject unsuitable for enrollment.
7. Has a history of any condition known to interfere with the absorption, distribution, metabolism or excretion of medication.
8. Has a history or presence of any clinically significant abnormality in vital signs, ECG or lab tests (except viremia, temperature and LFTs<3× the upper limit of normal) or that has any medical or psychiatric condition that may interfere with the study procedures or compromise subject safety.
9. Has participated in an interventional study and received study treatment within 30 days (or less than 5 half-lives of the investigational agent) prior to screening.
10. Has taken a dose of acetaminophen/paracetamol within 2 hours of screening.
11. Currently takes daily doses of a nonsteroidal anti-inflammatory drug (NSAIDS), such as aspirin, etoricoxib, celecoxib, ibuprofen, naproxen, diclofenac, meloxicam, etc.
12. Has taken a single dose of the following NSAIDs within the timeframes noted:
   ibuprofen or celecoxib: within 12 hours of screening,
   Aspirin, etoricoxib, diclofenac, meloxicam or naproxen: within 24 hours of screening
13. Is mentally or legally incapacitated.
14. Is a pregnant or breast-feeding woman.
15. Has an active gastroduodenal ulceration.
16. Has known hypersensitivity or another contraindication to etoricoxib or acetaminophen/paracetamol
17. Is an employee of the Sponsor, a relative of, or staff directly reporting to, the Investigator.

Subjects with laboratory test results which do not meet the above inclusion/exclusion criteria may have the underlying test repeated once if it is thought to represent a laboratory error, a reversible, clinically insignificant intermittent condition, or is not consistent with the subject's historical values. If inclusion/exclusion criteria are not met after the repeat test, the subject will not be enrolled in the study. Exceptions to the above eligibility criteria will not be granted.

Subject Withdrawal Criteria

Subjects may voluntarily withdraw from the study or be removed from the study at any time. A subject may be withdrawn at any time if it is determined that continuing in the study would result in a significant safety risk to the subject.

Premature withdrawal may occur for any of the following reasons:
Non-compliance with the protocol requirements
Pregnancy
Death
Adverse event (AE)
Subjects who demonstrate symptoms of severe dengue (severe abdominal pain, rapid breathing, persistent vomiting, blood in vomit, accumulation of fluid in the body, mucosal bleeding. Liver enlargement, rapid decrease in platelet count, lethargy, restlessness)
Subject request
Investigator request
Sponsor request

For subjects who are lost to follow-up (i.e., those subjects whose status is unclear because they fail to appear for study visits without stating an intention to withdraw), “due diligence” will be shown by documenting in the source documents all steps taken to contact the subject, e.g., dates of telephone calls, registered letters, etc.

All subjects prematurely discontinuing from the trial (prior to Visit 4/Day 7/EoS), regardless of cause, must be seen in the clinic for Visit 4/Day 7/EoS. Subjects who withdraw from the study prior to dosing a single tablet will return to the clinic for Visit 4/Day 7/EoS procedures and to return all unused study drug.

Prohibited Medications

Use of the following medications is prohibited for the duration of a subject's participation in the study from Screening/Visit 1 through EoS/Visit 4/Day 7:
Any investigational treatment other than etoricoxib.
All non-study associated analgesic medications taken for pain
The use of the following medications is prohibited throughout the study
   NSAIDs (including COX-2 selective inhibitors with the exception of the study drug)
   Opioids
   All cannabinoid containing products including cannabidiol oil topical analgesics such as diclofenac gel
   Gabapentin/pregabalin-containing products
   Benzodiazepines

Study Drug

Study drug refers to etoricoxib 120 mg and acetaminophen/paracetamol 750 mg. Etoricoxib is a tablet containing 120 mg etoricoxib. The tablet is pale green, apple shaped, biconvex debossed “204” on one side and “ACX 120” on the other. Etoricoxib is for oral administration only.

The study drug product is marketed by Merck and contains the following inactive excipients:
Core: Calcium hydrogen phosphate, croscarmellose sodium, microcrystalline cellulose
Coating: Carnauba wax, lactose monohydrate, hypromellose, titanium dioxide, triacetin, indigo carmine lake, yellow ferric oxide

Each dose of etoricoxib will consist of one tablet for the treatment of dengue fever:

etoricoxib 120 mg dose

Acetaminophen/paracetamol is marketed by Johnson & Johnson and contains the following inactive excipients:

Maize starch, pregelatinized maize starch, stearic acid

Each dose of acetaminophen/paracetamol 750 mg will consist of 1 tablet for the treatment of dengue fever:

Acetaminophen/paracetamol 750 mg dose

Study Drug Packaging and Labeling

All study drug (etoricoxib and acetaminophen/paracetamol) will be provided in bottles. The etoricoxib pack will include 7 tablets. The acetaminophen/paracetamol package will include 28 tablets. All study drug will be stored at room temperature. No preparation will be required.

Administration

The study drug will be dispensed to the subject following Randomization. Instructions for etoricoxib will direct the subject to take one tablet per day. Instructions for acetaminophen/paracetamol will direct the subject to take one tablet every 6 hours, for a total of 4 tablets per day. Subjects will be instructed to take study medication with approximately 8 ounces of water daily in the morning at approximately the same time of day as the FIRST treatment. Subjects assigned to the acetaminophen/paracetamol group will be instructed to take each subsequent dose 6 hours after the previous dose with approximately 8 ounces of water.

Pain Assessments:

Pain Right Now Assessment

The Primary endpoint is the change from baseline in the subject assessment of how much pain they have right now on a 0- to 10-point Numeric Rating Scale (NRS) measured three times daily and averaged over the first 48 hours of treatment after taking study drug. The subject will answer the following question regarding pain intensity as shown in Table 1. This questionnaire also supports an Exploratory endpoint measured over the 7-day treatment period.

TABLE 1
“Please rate your pain by marking the box under the number
that tells how much pain you have right now.”

| | | | | | | | | | | |
|---|---|---|---|---|---|---|---|---|---|---|
| 0 | 1 | 2 | 3 | 4 | 5 | 6 | 7 | 8 | 9 | 10 |

| | |
|---|---|
| No | Pain as |
| pain | bad as |
| at | you can |
| all | imagine |

This question is to be answered at 4 hours, 8 hours and 12 hours after the 1^{st} dose of study medication on Days 1 through 7. On Study Day 1, the time in between ratings may be 2, 3, or 4 hours depending on how early the subject took their first dose of study medication in the clinic.

Average Pain Assessment

Another Secondary endpoint evaluates the efficacy of etoricoxib 120 mg versus acetaminophen/paracetamol 3 g daily in subjects with dengue fever as measured by the subject assessment of the daily average pain intensity over the first 48 hours. This questionnaire as shown in Table 2 also supports an Exploratory endpoint measured over the 7-day treatment period.

TABLE 2
“Please rate your pain by marking the box under the number that
best describes your pain on the average in the last 24 hours”

| | | | | | | | | | | |
|---|---|---|---|---|---|---|---|---|---|---|
| 0 | 1 | 2 | 3 | 4 | 5 | 6 | 7 | 8 | 9 | 10 |

| | |
|---|---|
| No | Pain as |
| pain | bad as |
| at | you can |
| all | imagine |

This question will be answered daily in the pain diary on Days 1 through 7.

Worst Pain Assessment

Another Secondary endpoint is the change from baseline in the average of the daily worst pain intensity scores on a 0- to 10-point NRS measured at bedtime and averaged over the first 48 hours of treatment. This questionnaire as shown in Table 3 also supports an Exploratory endpoint measured over the 7-day treatment period.

TABLE 3
“At bedtime, please rate your worst pain over the
last 24 hours. Please rate your pain by marking the
box under the number that best describes your pain at its worst
in since taking your 1^{st} dose of study medicine.”
Time you are recording your pain:.

| | | | | | | | | | | |
|---|---|---|---|---|---|---|---|---|---|---|
| 0 | 1 | 2 | 3 | 4 | 5 | 6 | 7 | 8 | 9 | 10 |

| | |
|---|---|
| No | Pain as |
| pain | bad as |
| at | you can |
| all | imagine |

This question will be answered daily in the subject diary at bedtime on study Days 1 through 7.

Pain Locations:

Subjects will also be asked to locate the sources of their pain daily based on the following question in the Subject Diary as shown in Table 4.

TABLE 4
“At bedtime, record where you felt pain associated with dengue
fever today. In the chart below, mark the box besides all the
locations where you felt pain from dengue fever today.”

| | |
|---|---|
| | Mark which pain you felt |
| Location of Pain | today with an “X” |

Headache
Pain behind the eyes/eye pain
Joint pain
Muscle pain
Back pain
Stomach pain

Temperature Assessments

A Key Secondary Endpoint is the change from baseline in the time-weighted average (area under the curve divided by time interval) temperature over the first 48 hours of treatment as measured by continuous fever monitor readings and digital thermometer readings 4 times per day.

Subjects will also be instructed to take their temperature with the infrared thermometer 4 times a day and to record it in their subject diary to provide the digital thermometer readings for this endpoint. If the subject takes a bath, they will also be asked to record their temperature prior to and after taking a bath as shown in Table 5 and Table 6.

TABLE 5
“You need to record your temperature today 4 times today, in
the morning, around noon, in the evening around dinnertime, and
just before you go to bed. Please take your temperature with the
digital infrared thermometer and record it in the table below.
Please write down the temperature and the time it was taken.”

| | |
|---|---|
| Time you took your temperature | Record the temperature displayed on |
| (indicate AM or PM) | the digital infrared thermometer |

TABLE 6
Baths
“If you take any baths today, please record the time you took the
bath. You also need to record your temperature before you get into
the bath and then again when you get out of the bath. Use the digital
infrared thermometer to take the temperature. This is in addition
to the four times you take your temperature during the day.”

| | | |
|---|---|---|
| Time you took a bath | Temperature before you | Temperature when you |
| (indicate AM or PM) | entered the bath | got out of the bath |

Efficacy and Safety Parameters

Primary efficacy will be evaluated by calculating the change from baseline in the average of the subject assessment of how much pain they have right now on a 0- to 10-point NRS over the first 48 hours of treatment. The subjects will record their score on how much pain they have three times per day.

The key secondary endpoint is the time weighted average difference (area under the curve divided by time interval) in temperature from baseline over the first 48 hours of treatment. Other secondary endpoints include the change versus baseline in the mean of the daily average pain intensity score over the first 48 hours, the change versus baseline in the subject's assessment of worst pain on a 0- to 10-point NRS once a day at bedtime and the change from baseline in AST and ALT at 72 hours and at the end of the study. Safety will be monitored with physical examinations, vital signs, ECGs, clinical laboratory testing, and AE/SAE assessments.

An exploratory endpoint is the time weighted average difference in viral load (area under the log₁₀ transformed plasma viremia curve from day 1 to the end of the study divided by the time interval) compared to baseline. Standardization of data capture is provided in detail in the remainder of this section. Assessments will be performed in relation to dosing as indicated.

Medical History/Concomitant Medications

General, relevant medical history will be recorded on the CRF and will include information relating to any prior or existing medical conditions involving the following disease types or systems: infectious diseases, allergic, metabolic/endocrine/nutritional, hematopoietic, musculoskeletal, dermatologic, head, eyes, ears, nose and throat (HEENT), breasts, respiratory, cardiovascular, gastrointestinal/hepatic, genitourinary/renal, neurological, and psychiatric/psychosocial. Subjects with ongoing significant major organ disease will be excluded. Concomitant medications for pain during the 3 months prior to study enrollment and concomitant medication used to treat other medical conditions during the last 30 days will be recorded on the CRF.

Demographics

During Screening/Visit 1, subject initials, age, gender, ethnicity and race will be recorded on the CRF.

Physical Examination

A complete physical examination will be performed during Screening and Visit 4/Day 7/EOS. A complete physical examination of all body systems will include the following: general appearance, skin, HEENT, heart, lymph nodes, lungs, abdomen, extremities/joints, neurological systems, and mental status. For all other visits, a brief symptom related physical examination will be performed if indicated by an AE. Height and weight will be measured at Screening and at Visit 4/EoS/Day 7.

Vital Signs

Vital signs will be recorded at each clinic visit, measured after at least 5 minutes rest, will include seated systolic blood pressure (SBP), diastolic blood pressure (DBP), heart rate (HR), respiratory rate (RR), and body temperature. All vital sign measurements will be performed by appropriately qualified and authorized study personnel, using appropriate equipment.

Blood pressure (BP) will be measured after at least 5 minutes rest and if possible, in the same arm at each visit by using an automated sphygmomanometer. The results will be recorded in millimeters of mercury (mmHg). HR will be measured in the radial artery in the dominant arm for 30 seconds and will be recorded as beats per minute (bpm). RR will be measured and recorded in breaths per minute. Body temperature (oral measurement) will be measured in degrees Celsius using a digital thermometer.

ECG

A standard, digital 12-lead ECG will be obtained after at least 5 minutes rest at Screening and Visit 4/EoS/Day 7. A trained ECG technician will perform the ECGs and all ECG results must be reviewed at the site by the Investigator or a medically qualified designee for clinical management of the subject. Abnormal findings will be identified as either clinically significant (CS), or not clinically significant (NC S). CS findings are to be reported as AEs. ECG reports will include rhythm, rate, axis, PR, QRS, and corrected (by both Fridericia and Bazett) and uncorrected QT intervals.

Clinical Laboratory

Samples of blood and urine will be collected for clinical laboratory tests at Visit 1/Screening and Visit 4/EOS/Day 7

Tests will be conducted as designated below and processed by a local laboratory, except for the sample for the Viral Titer test, which will be processed by a biomolecular laboratory:
Hematology: White blood cell (WBC) count with differential (neutrophils, lymphocytes, eosinophils, monocytes, basophils), hemoglobin, hematocrit, platelet count, and red blood cell (RBC) count.
Serum Chemistry Profile: Albumin, alkaline phosphatase (AP), alanine aminotransferase (ALT), aspartate aminotransferase (AST), blood urea nitrogen (BUN), calcium, chloride, bicarbonate, creatinine, glucose, phosphate, potassium, sodium, total bilirubin, total protein.
Urinalysis: Protein, glucose, nitrite, ketones, blood (hemoglobin), pH, specific gravity, microscopic bacteria, RBCs, WBCs, casts, crystals, and cells. Microscopic examination of sediment will be performed only if the results of the urinalysis dipstick evaluation are positive.
Viremia: The virus titer will be measured via quantitative PCR. Blood samples for this test will be sent to a biomolecular laboratory in Guatemala for analysis. Blood samples must be frozen at −70° C. and sent to biomolecular lab.

Samples of blood will be obtained at Visit 2 (Day 3) and Visit 3 (Day 4) for the following tests:
Alanine aminotransferase (ALT): To be processed by local laboratory
Aspartate aminotransferase (AST): To be processed by local laboratory
Viremia: The virus titer will be measured via quantitative PCR. Blood samples for this test will be sent to a biomolecular laboratory in Guatemala for processing. Blood samples must be frozen at −70° C. and sent to biomolecular lab).

Abnormal and Clinically Significant Results

All abnormal hematology, chemistry, and urinalysis laboratory values will be categorized as either clinically significant (CS) or non-clinically significant (NCS). Clinical significance is defined as any variation in laboratory parameters, which has medical consequences that result in an alteration in the subject's medical care. The WHO Toxicity Criteria as shown in FIG. 5 will be used as a guide when evaluating the clinical significance of all abnormal clinical laboratory results. In case of CS laboratory results, the subject will be monitored with additional laboratory assessments until (1) values have reached normal range and/or baseline levels, or (2) it has been judged that the abnormal values are not related to the administration of study drug or other protocol-specific procedures.

Dosing Instructions

The first dose of study drug will be administered in the clinic following Randomization. All subsequent dosing will be done outpatient. Study drug will be administered at approximately the same time of day each day with the first dose administered in the morning with the possible exception of the first dose which will depend on the timing of the screening randomization visit (Visit 1/Day 1). Subjects will record the how much pain they have right now at the time of dosing on a 0- to 10-point NRS (where 0=none and 10=pain as bad as you can imagine).

Subject Diary

Subjects will be trained on the use of the diary at Screening and retrained during the study as required. Subjects will record the date and time of taking a dose of the study drug. Efficacy data will be collected in the diary. Subjects will record their assessment of how much pain they have right now on a 0- to −10-point NRS three times per day, in the morning, afternoon and bedtime. Subjects will be asked “Please rate your pain by marking the box under the number that tells how much pain you have right now. Zero is no pain. 10 is pain as bad as you can imagine.”

Subjects will record their worst pain intensity on a 0- to 10-point NRS once a day at bedtime. Subjects will be asked to “Please rate your pain by marking the box under the number that best describes your pain at its worst in the last 24 hours. Zero is no pain and 10 is pain as bad as you can imagine.”

Subjects will record their average pain intensity on a 0- to 10-point NRS once a day at bedtime. Subjects will be asked “Please rate your average pain by marketing the box under the number that best describes your average pain over the last 24 hours. Zero is no pain and 10 is pain as bad as you can imagine.”

Subjects will be asked to record their temperature 4 times a day with the digital infrared thermometer: When they get up, noon, evening and bedtime. They will also be asked to record when they take baths if their temperature rises above 38 degrees C., and their temperature before and after the bath. Subjects will also record concomitant medications and location of pain on a daily basis in their diary.

Adverse Events and Serious Adverse Events

Adverse Events (AE)

An AE is defined as any undesirable physical, psychological, or behavioral effect experienced by a subject during his/her participation in an investigational study, in conjunction with the use of the drug, whether or not product related. The occurrence of AEs will be sought by non-directive questioning of the subject at each visit in the study. AEs may include, but are not limited to:
Subject gives or objective symptoms are spontaneously offered by the subject and/or are observed by medical staff, and
Changes in laboratory abnormalities that are clinically relevant as assessed and for which a medical intervention was initiated.

Subjects will be counselled to call the clinic if they experience any unusual symptom on a non-clinic day. Signs, symptoms, and/or laboratory abnormalities already existing prior to the use of the product are not considered AEs after treatment unless they reoccur after the subject has recovered from the pre-existing condition or they represent a clinically significant exacerbation in intensity or frequency. AEs are collected from the time the subject signs the informed consent form until the completion of Visit 4/EoS/Day 7. AEs reported prior to dosing will be captured and considered non-treatment emergent AEs. This includes any laboratory AE generated from laboratory tests which were performed at screening as well as follow-up laboratory evaluations for laboratory AEs identified prior to dosing.

All AEs must be recorded in the site's study records and the AE CRF with the following information:
1. Relationship to Study Drug: It will be assessed whether an AE is considered to be drug-related. In assessing this relationship, information will be used about the drug as outlined in the Investigator's Brochure (IB), the subject's pre-existent medical conditions/concurrent medication, and chronology of the event relative to drug administration. The following definitions will be used:
   Reasonably or possibly related applies to those AEs that, after careful medical consideration at the time they are evaluated, are considered to have at least a possible relationship to study drug.
   Not reasonably or not possibly related applies to those AEs that, after careful medical consideration at the time they are evaluated, are considered to have no relationship, or no reasonable possibility of a relationship, to study drug.
2. Event Severity: The severity of the AE will be assessed using the WHO Toxicity Criteria, as shown in FIG. 5. The WHO criteria assign a grade of 1 through 4 to indicate the severity of AEs. For AEs that are not listed in the WHO criteria, professional medical judgment will be used to assess the severity of the AE. The following are guidelines to be used to judge the event severity of an AE that is not in the WHO Toxicity Criteria:
   Mild—awareness of sign or symptom, but easily tolerated
   Moderate—discomfort enough to cause interference with usual activity
   Severe—incapacitating with inability to work or perform usual activity
   Life Threatening
3. Duration: Start and end dates and times, or if continuing.
4. Action taken.
5. Whether it constitutes a SAE, per definition below.
6. Outcome: resolved, resolved/with sequelae, continuing, death, or unknown (only for subjects that are lost to follow-up).

An attempt will be made to establish a diagnosis of the AE based on signs, symptoms and/or other clinical information. In such cases, the diagnosis, and not the individual signs/symptoms or laboratory abnormalities, will be documented in the subject's source documentation and the CRF unless the etiology of the event is unknown. An assessment will be made at each visit (or more frequently if necessary) of any changes in severity, the suspected relationship to study drug, the interventions required to treat it and the outcome.

Treatment Emergent Adverse Events (TEAE)

A TEAE will be an AE that occurred during the study after the first dose of study drug or that was present prior to dosing and exacerbates after the first dose of study drug.

Serious Adverse Events (SAE)

An SAE is any AE that results in any of the following outcomes:
Death: This includes death unrelated to the study drug (e.g., car accident). If a subject dies during the study and an autopsy is performed, autopsy results will become part of the subject's study chart and a copy will be sent to the Sponsor.
Life-threatening experience
Required or prolonged inpatient hospitalization: Exceptions will be hospitalizations for a) elective or preplanned treatment for a pre-existing condition that is unrelated to the indication under study and has not worsened since the start of study drug or b) treatment on an emergency outpatient basis for an event not fulfilling any of the definitions of a SAE and not resulting in hospital admission
Persistent or significant disability/incapacity
Congenital anomaly
Important medical events that may not result in death, be immediately life threatening, or require hospitalization may be considered a SAE when, based upon medical judgment, they may jeopardize the subject and may require intervention to prevent one of the outcomes listed above.

Statistics

Prior to the start of the study, a detailed statistical analysis plan will be developed, and decisions will be made regarding the integrity of subject data for inclusion in the statistical analysis. All tests of treatment effects will be conducted at a one-sided alpha level of 0.05. The primary objective of the study is to evaluate the efficacy of etoricoxib versus acetaminophen/paracetamol 3 g daily in subjects with dengue fever. Data from all investigative sites will be pooled for all planned analyses. Analysis of individual site findings will be considered if necessary. For those measures that are analyzed using change from baseline scores, observed scores may also be presented descriptively. Any changes in the data analysis methods described in the protocol will require an amendment only if it changes a principal feature of the protocol. Any other changes to the data analysis methods described in the protocol and the justification for making the change will be described in the statistical analysis plan as well as the clinical study report. Additional exploratory analyses will be conducted as deemed appropriate. All data listings, summaries and analyses will be performed by Cytel.

Sample Size

This Phase IV Pilot study is designed to evaluate the efficacy of etoricoxib 120 mg versus acetaminophen/paracetamol in subjects with dengue fever. An interim analysis (IA) for potential sample size increase is planned after pain intensity data are available from approximately N=16/group. The purpose of the IA is to verify that the design can yield a statistically supported conclusion based on the planned sample size of N=20 per group. If not, the trial can either be stopped for a futility conclusion or the sample size may be increased up to N=40 per group to boost statistical power to 80% or as close to 80% as possible with maximum N=40 per group. The IA decision to increase the sample size is based on conditional power according to the adaptive sample size re-estimation design by Mehta & Pocock (2011). For TRUE underlying mean difference between treatments in pain intensity reduction of 1.5 & standard deviation (SD)=2, there is approximately 80% overall power (alpha=0.05 1-sided), and 91% power if the IA result is promising. The minimum OBSERVED difference that achieves statistical significance is 1.04.

Detailed Size Re-Estimation Design and Performance Characteristics

A randomized pilot trial comparing etoricoxib to acetaminophen/paracetamol for pain and fever effect in dengue fever is being conducted. The primary endpoint is pain intensity reduction from baseline. The sample size of N=20 per treatment group is considered adequate since an OBSERVED mean difference between treatments of 1 unit on the 0- to 10-point NRS pain scale is considered potentially minimally clinically important (assumes SD˜2 and alpha=0.05 1-sided). Since the signal-to-noise ratio in this disease is uncertain, an interim analysis for potential sample size re-estimation is considered.

The proposed design has a planned total sample size of N=20 per treatment group with an interim analysis (IA) after data from the first 16 subjects per treatment group become available. The purpose of the IA is to potentially increase the sample size up to a maximum of N=40 per treatment group if needed to increase the chance of obtaining a statistically significant difference between treatments. The increase in sample size is based on conditional power (CP). CP is the probability of achieving a statistically significant (alpha=0.05 1-sided in this case) difference between treatments at the final analysis if the TRUE underlying mean difference is equal to that OBSERVED at the IA. This is the “promising” zone adaptive design by Mehta & Pocock (2011). The following decision algorithm is considered:
(futility zone) If the observed difference in pain reduction (etoricoxib versus acetaminophen/paracetamol) is ≤0, the trial may be stopped for futility
(unfavorable zone) If the observed difference >0 and CP<50%, the trial continues to planned N=20/group for final analysis, since it is not worthwhile to increase the sample size, but the IA results are not so bad as to suggest to stop the trial for futility
(promising zone) If 50%≤CP<80%, sample size is increased to that with CP=80% or as close to 80% as possible with maximum N=40/group
(favorable zone) If CP≥80%, the trial continues to N=20/group for final analysis
There is no efficacy zone in this example to stop the trial at the IA to support an efficacy conclusion in order to retain the full type 1 error for the final analysis.

The final analysis is carried out based on the usual ANCOVA or mixed model repeated measures (MMRM) analysis with type 1 error controlled at 0.05 1-sided (Chen, DeMets & Lan, 2004). This design was simulated 10,000 times for potential TRUE underlying differences in mean 0-10 NRS pain reduction 1, 1.3, and 1.5 (SD=2). Performance Characteristics are summarized in the Table 6 below. Note that for TRUE underlying difference 1.0, this design has only 48% power overall; however, if IA results are promising (i.e., CP at least 50%, but less than the targeted 80%, which occurs with probability 0.12), then sample size is increased, and power becomes 77%. If the IA yields a result in the favorable zone (which occurs with probability 0.36), there is at least 80% power, by definition. There is 0.08 probability of the IA yielding a futility zone result, and 0.44 of yielding an unfavorable zone result. However, the minimum OBSERVED difference that would yield statistical significance (alpha=0.05 1-sided) at the N=20/group final sample size is 1.04, which is approximately equal to a potential minimum clinically important difference on the 0- to 10-point NRS scale. If the sample size is increased to N=30/group, the minimum observed difference that would yield statistical significance is 0.85, and with N=40/group, is 0.74. Corresponding values for other potential IA outcomes and other potential TRUE underlying mean differences are in the rest of Table 6. Note that if a non-adaptive design were employed, power would be only 48% with N=20/group and with no chance of increasing N via IA.

TABLE 6
Power and Sample Size Estimates

| TRUE underlying difference in 0- to | | | |
|---|---|---|---|
| 10-point NRS pain improvement | 1.0 | 1.3 | 1.5 |
| Power* for non-adaptive alternative (N = 20/group) | 0.48 | 0.66 | 0.77 |
| Power* for non-adaptive alternative (N = 40/group) | 0.72 | 0.9 | 0.96 |
| Overall Power* of Adaptive SSR Design | 0.52 | 0.69 | 0.79 |
| Power* if IA “Promising” | 0.77 | 0.85 | 0.91 |
| Probability IA Futility | 0.08 | 0.03 | 0.02 |
| Probability IA unfavorable | 0.44 | 0.31 | 0.24 |
| Probability IA “Promising” | 0.12 | 0.14 | 0.12 |
| Probability IA Favorable | 0.36 | 0.52 | 0.62 |
| Expected (“average”) Total Sample | 42 | 42 | 42 |
| Size (2 groups combined) | | | |
| Expected (“average”) Total Sample | 58 | 58 | 57 |
| Size if IA “Promising” | | | |

SSR = sample size re-estimation
*alpha = 0.05 1-sided

Randomization

This is a two-center, randomized, open-label, active comparator-controlled parallel-group study for 7 days. Subjects will be centrally randomized to one of 2 treatment arms to receive etoricoxib 120 mg or acetaminophen/paracetamol 3 g per day (in a 1:1 ratio).

Analysis Populations

Primary Efficacy

The statistical analysis will be based on the analysis populations as defined below:

| | |
|---|---|
| Safety | All randomized subjects who use at least one dose of study |
| population | drug, regardless of whether or not they undergo any study |
| | assessments. Subjects are evaluated by the drug they use, |
| | not by the drug to which they are randomized. |
| Full Analysis | All randomized subjects who use at least one dose of study |
| Set (FAS) | drug and have any post-dose assessments. Subjects are |
| population | evaluated by the drug to which they are randomized |
| PP | All ITT subjects will be considered per protocol (PP) if they |
| population | dose and do not deviate from the protocol. |

A list of violations to the protocol which would exclude subjects from the PP population will be identified prior to locking the database.

Efficacy Analyses

All the efficacy analyses will be performed on the FAS population. The PP population will be used as supportive analyses. The primary endpoint measured in each subject is the change from baseline in the average of the subject assessment of the pain they have right now on a 0- to 10-point NRS measured three time daily over the first 48 hours of treatment. The mean of these per subject averages will be compared between treatments using an analysis of covariance (ANCOVA) model including factors for baseline pain, baseline temperature, and treatment. The null hypothesis is that the population mean of the etoricoxib group is greater than or equal to that of the acetaminophen/paracetamol group. The alternative hypothesis is that the population mean of the etoricoxib group is less than that of the acetaminophen/paracetamol/paracetamol group. The null hypothesis will be tested via t-test derived from the least squares means from the ANCOVA model fit. Missing values will not be imputed for subjects with at least 1 pain observation on each of the first 2 treatment days. Missing values for subjects with no pain observations on one or both of the first 2 treatment days will be imputed via multiple imputation from their respective treatment group unless they dropout from the study for a treatment-related reason, in which case their missing data will be imputed from the acetaminophen/paracetamol group.

The key secondary endpoint will be analyzed similarly as the primary endpoint. Other efficacy endpoints will be analyzed similarly except with no imputation for missing data. Details of the efficacy analyses will be in the SAP.

Safety Analyses

Values for all safety variables will be listed by subject and visit.

Where appropriate, safety variables will be summarized by using descriptive statistics, separated by treatment arm, dose, and time of assessment. Descriptive statistics for quantitative variables will include n, mean, median, minimum, maximum, and standard deviation. Descriptive statistics for qualitative variables will include frequency counts and percentages. Confidence intervals will be provided for AEs of special interest.

Adverse events of special interest include the incidence of events listed below as well as the incidence of discontinuation due to these AEs. The AEs of special interest are as follows:

Liver function

Hepatitis

Bleeding events

Hypertension

Upper gastrointestinal perforations, ulcers, or bleeds

Thrombotic cardiovascular events

Adverse Events

AEs will be coded by the Medical Dictionary for Regulatory Activities (MedDRA) preferred terms, and all summary tables for AEs will be organized by these categories. Frequency counts and percentages will be presented for subjects with AEs within each system organ class and preferred term, separated by treatment arm. Both subjects ever experiencing an event as well as total events will be presented. Descriptive statistics will also be calculated for each treatment arm and treatment sequence and dose (initial and second dose for recurrence or rescue medication) for AE relationship and AE severity. If multiple intensities are reported for a given AE for a subject, the most severe intensity will be counted. A separate, similar analysis will be conducted for TEAEs. An AE with the date of onset after a dose of study drug, or an event that worsens in intensity after a dose of study drug will be considered a TEAE. SAEs and TEAEs that resulted in termination of the study drug and withdrawal from the study will be presented.

Physical Examinations, Vital Signs, ECG Parameters and Clinical Laboratory Test Values

By-subject listings of physical examinations, vital signs, ECG parameters and clinical laboratory data will include indications of values that are outside the reference ranges, and values that are clinically significant. Shift tables describing out-of-reference range shifts will be provided for vital signs, ECGs and clinical laboratory test results from the Screening to Visit 4/EoS/Day 7, as appropriate by treatment arm and dose.

### REFERENCES FOR EXAMPLE 1

1. UK Summary of Product Characteristics
2. Lin C-K, Tseng C-K, Wu Y-H, et al. Cyclooxygenase-2 facilitates dengue virus replication and serves as a potential target for developing antiviral agents. Vol 72017
3. Dengue, Guidelines for Diagnosis, Treatment, Prevention and Control, 2009, A joint publication of the World Health Organization (WHO) and Training in Tropical Diseases (TTD),
4. Dengue: Symptoms and Treatment, Centers for Disease Control, www.cdc.gov.dengue, page last updated Sep. 26, 2019
5. WHO Fact Sheet, 2019, www.who.int.dengue and severe dengue, last updated Oct. 4, 2019
6. LiverTox: Clinical and Research Information on Drug Induced Liver Injury: Acetaminophen/paracetamol. www.livertox.nih gov, updated Jul. 1, 2019
7. Kellstein, D, Fernandes L, Symptomatic Treatment of dengue, should the NSAID contraindication be reconsidered? Postgrad Med., 2019 March; 131(2): 109-1116
8. Wu W L, Ho L J, Chang D M, Chen C H, Lai J H. Triggering of DC migration by dengue virus stimulation of COX-2-dependent signaling cascades in vitro highlights the significance of these cascades beyond inflammation. Eur J Immunol. 2009; 39(12):3413-3422
9. Hideri, K, Suzuki T, Dengue Virus Receptor, Tropical Medicine and Health, 39 (4), 2011, pp 37-43
10. WHO: Immunization, Vaccine and Biologicals, Dengue Fever: www.who.int.immunization.diseases.dengue. last updated Jul. 29, 2016
11. Pandejpong D, Saengsuri P, Rattarittamrong R, Rujipattanakul T Chouriyagune C. Is excessive acetaminophen/paracetamol intake associated with transaminitis in adult patients with dengue fever? Intern Med J. 2015; 45: 653-658
12. Thomas L, Brouste Y, Najioullah F, et al. Predictors of severe manifestations in a cohort of adult dengue patients. J Clin Virol. 2010; 48: 96-99
13. Djossou F, Vesin G, Walter G, et al. Incidence and predictive factors of transaminase elevation in patients consulting for dengue fever in Cayenne Hospital, French Guiana. Trans R Soc Trop Med Hyg. 2016; 110: 134-140
14. Vasikasin, V, Rojdumrongrattana T, Chuerboonchai W, Siriwattana T, Effect of standard dose paracetamol versus placebo as Antipyretic therapy on liver injury in adult dengue infection: a multicenter randomised controlled trial. The Lancet Global Health 2019; 7: e664-70
15. Ranganathan S S, Sathiadas M G, Sumanasena S, Fernandopulle M, Lamabadusuriya S P, Fernandopulle B M, Fulminant hepatic failure and paracetamol overuse with therapeutic intent in febrile children. Indian J Pediatr. 2006; 73: 871-875
16. Tan S S, Bujang M A, The clinical features and outcomes of acute liver failure associated with dengue infection in adults: a case series. Braz J Infect Dis. 2013; 17: 164-169
17. Hardin H. Etoricoxib: A New COX-2 Inhibitor. Pharma Note. 2004; 20(3)
18. Etoricoxib Patient Information, Guatemalan Ministry of Health
19. Schwartz J I, Chan C C, Mukhopadhyay S, Cuyclooxygenase-2 inhibition by rofecoxib reverses naturally occurring fever in humans. Clin Pharmacol Therapy 1999: 65:653-60
20. Rawal, N., Viscusi, E., et al. Evaluation of etoricoxib in patients undergoing total knee replacement surgery in a double-blind, randomized controlled trial. BMC Musculoskeletal Disorders 2013; 14:300.
21. Viscusi, E. R., et al. Perioperative use of etoricoxib reduces pain and opioid side-effects after total abdominal hysterectomy: a double-blind, randomized, placebo-controlled phase III study. Current Medical Research and Opinion 2012, 28(8), 1323-1335.
22. Rasmussen, G, Malmstrom K, Bourne, M, Jove, M, Rondeau S, Kotely P, Ang J, Aversano M, Recin A, Etoricoxib provides analgesic efficacy to patients after knee or hip replacement surgery: A randomized, double-blind placebo-controlled study. Anesthesia and Analgesia 2005: 101(4): 1104-1111 1104-1111
23. Patriganani P, Capone M C, Tecconelli S, Clinical pharmacology of etoricoxib: a novel selective COX-2 inhibitor, Expert Opinion Pharmacotherapy, 203; 4(2): 265-84
24. Dallob A, Hawkey C J, Greenberg H, Wight N, De Schepper P, Waldman S, Wong P, DeTora L, Gertz B, Agrawal N, Wagner J, Gottesdiener K. Characterization of etoricoxib, a novel, selective COX-2 inhibitor. J Clin Pharmacol. 2003; 43:573-585
25. Cannon, C. P., Curtis, S. P., FitzGerald, G. A., Krum, H., Kaur, A., Bolognese, J. A., Laine, L. Cardiovascular outcomes with etoricoxib and diclofenac in patients with osteoarthritis and rheumatoid arthritis in the Multinational Etoricoxib and Diclofenac Arthritis Long-term (MEDAL) programme: a randomised comparison. The Lancet 2006, 368(9549), 1771-1781.
27. Daniels S E, Bandy D P, Christensen S E, Boice J, Losada M, Liu H, Mehta A, Peloso P M, Evaluation of the dose range of etoricoxib in an acute pain setting use the post operation dental pain model, Clinical J Pain, 2011, January; 27(1):1-8
28. Malmstrom K, Sapre A, Coughlin H, Agrawal N G, Mazenko R S, Fricke J R, Etoricoxib in acute pain associated with dental surgery; a placebo and active comparator-controlled dose ranging study; Clin Ther. 2004, 26(5) 667-679
29. Malstromm K, Kotey P, Coughlin H, Desjardins P, A randomized, D\double-blind, parallel-group study comparing the analgesic effect of etoricoxib to placebo, naproxen sodium and acetaminophen with codeine using the dental impaction pain model, Clin J Pain, 2004; 20(3):147-55
30. Gammaitoni A, Viscusi E, Hartrick C, Frenkl T, Peloso P, Ko A, Morgan L, Mehta A, Papanicolou D, Perioperative use of etoricoxib in patients undergoing total abdominal hysterectomy, Journal of Pain, 2011: 12(4): p4
31. Tsukas C, Eyster E, Shingo S, Mukhapadhyay S, Giallella K, Curtis S, Reicin A, Melian A, Evaluation of the efficacy and safety of etoricoxib in the treatment of hemophilic arthropathy. Blood 2006; 107(5): 1785-1790
32. James L P. Acetaminophen. In, Kaplowitz N, DeLeve L D, eds. Drug-induced liver disease. 3rd ed. Amsterdam: Elsevier, 2013, pp. 331-42.
33. Larson A M, Polson J, Fontana R J, et al. Acetaminophen induced acute liver failure: results of a United States multicenter, prospective study. Hepatology. 2005; 42(6): 1364-1372.
34. Fernando S, Wijewickrama A, Gomes L, Punchewa C T, Madusanka S D, Dissanayake H, Jeewandara C, Patterns and Causes of Liver Involvement in Acute Dengue Infection, BMC Infec Dis, 2016, Jul. 8, 16:319
35. Bower W A, Johns M, Margolis H S, Williams I T, Bell B P, Population Based Surveillance for Acute Liver Failure, Am J Gastroenterol, 2007: 102(11): 2459-63
36. Etoricoxib Product Characteristics, EU
37. Nourjah P, An Ahmade S R, Karwaski C, Estimates of Acetaminophen (paracetamol) associated overdoses in the United States. Pharmacoepidemiol Drug Safe. 2006; 15(6):398-405
38. Mehta, C R and Pocock S J (2011). Adaptive Increase in Sample Size when Interim Results are Promising: A Practical Guide with Examples. Statistics in Medicine30(28): 3267-84
39. Chen J Y H, DeMets D L, Lan K K G. (2004). Increasing the sample size when the unblinded interim result is promising. Statistics in Medicine, 23(7), 1023-1038
40. EAST 6.5 User Manual, Cytel Inc (2018), Cambridge Mass.

Note on Scope of the Present Disclosure

The above are descriptions and preferred embodiments are examples only and are in no way intended to limit the potential combinations under which the present disclosure could be utilized.

What is claimed is:

1. A method for treating one or more symptoms in a subject having viral hemorrhagic fever (VHF), the method comprising administering to the subject a pharmaceutical composition comprising a therapeutically effective amount of etoricoxib or a pharmaceutically acceptable salt thereof.

2. The method of claim 1, wherein the pharmaceutical composition further comprises a pharmaceutically acceptable carrier.

3. The method of claim 1, wherein the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

4. The method of claim 1, wherein the VHF is caused by one or more RNA viruses derived from a family of viruses selected from Arenaviridae, Bunyaviridae, Filoviridae, Flaviviridae, and any combinations thereof.

5. The method of claim 4, wherein the VHF is caused by one or more RNA viruses from the Flaviviridae family of viruses.

6. The method of claim 5, wherein the VHF is caused by a Dengue Virus.

7. The method of claim 1, wherein the therapeutically effective amount of the etoricoxib causes a reduction in viral load, viral titer, or viral shedding.

8. The method of claim 1, wherein the pharmaceutical composition is administered at a duration and a dosage such that the subject experiences a reduction in the one or more symptoms.

9. The method of claim 8, wherein the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

10. The method of claim 1, wherein the pharmaceutical composition is provided in a quantity sufficient for a single course of therapy.

11. The method of claim 1, wherein the pharmaceutical composition is provided in a single course of therapy package.

12. The method of claim 1, wherein the pharmaceutical composition is administered at a dosing schedule matched to the time course of the VHF.

13. The method of claim 12, wherein the pharmaceutical composition comprises a dose of etoricoxib sufficient to achieve maximum anti-pyretic effect during the febrile phase of the infection.

14. The method of claim 13, wherein the method further comprises administering to the subject a lower maintenance dose during the critical phase and/or recovery phase of the infection compared to the dose administered during the febrile phase of the infection.

15. The method of claim 1, wherein the pharmaceutical composition comprising etoricoxib is administered at a dose that does not affect platelet aggregation or bleeding time.

16. The method of claim 1, wherein the pharmaceutical composition is administered at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

17. The method of claim 1, wherein the pharmaceutical composition is administered as an initial loading dose of about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

18. The method of claim 17, wherein the initial loading dose is administered during the febrile phase of the infection.

19. The method of claim 17, further comprising administering a maintenance dose of etoricoxib at about 30 mg/day, about 40 mg/day, about 50 mg/day about 60 mg/day, about 70 mg/day, about 80 mg/day, about 90 mg/day, about 100 mg/day, about 110 mg/day, or about 120 mg/day.

20. The method of claim 19, wherein the maintenance dose is administered for up to 3 days, up to 5 days, up to 7 days, up to 10 days, or up to 14 days.

21. The method of any one of claims 19-20, wherein the maintenance dose is administered during the critical phase and/or recovery phase of the infection.

22. The method of claim 19, wherein the maintenance dose is administered until the subject experiences a reduction in the one or more symptoms or until the one or more symptoms resolve.

23. The method of claim 1, wherein the pharmaceutical composition is suitable for oral administration.

24. The method of claim 23, wherein the pharmaceutical composition comprises a solid oral dosage formulation.

25. The method of claim 24, wherein the solid oral dosage formulation is a tablet.

26. The method of claim 24, wherein the solid oral dosage formulation is a capsule.

27. The method of any one of claims 25-26, wherein the solid oral dosage formulation is provided in a blister pack container.

28. The method of claim 23, wherein the pharmaceutical composition comprises an oral suspension.

29. The method of claim 23, wherein the pharmaceutical composition comprises a liquid solution.

30. The method of claim 1, further comprising administering a second pharmaceutical composition comprising an active agent or a pharmaceutically acceptable salt thereof, wherein the active agent is not etoricoxib.

31. The method of claim 30, wherein the active agent or a pharmaceutically acceptable salt thereof is acetaminophen (paracetamol).

32. The method of claim 1, wherein the method does not increase the risk of an adverse event as compared to administration of acetaminophen to the subject.

33. The method of claim 32, wherein the adverse event is a bleeding event.

34. The method of claim 33, wherein the bleeding event is gastrointestinal bleeding.

35. The method of claim 32, wherein the adverse event is an adverse cardiovascular event.

36. The method of claim 35, wherein the adverse cardiovascular event is cardiothrombosis.

37. The method of claim 1, wherein the method does not increase the rate of an adverse event as compared to administration of acetaminophen to the subject.

38. The method of claim 37, wherein the adverse event is a bleeding event.

39. The method of claim 38, wherein the bleeding event is gastrointestinal bleeding.

40. The method of claim 37, wherein the adverse event is an adverse cardiovascular event.

41. The method of claim 40, wherein the adverse cardiovascular event is cardiothrombosis.

42. The method of claim 1, wherein the method results in fewer adverse events as compared to administration of acetaminophen to the subject.

43. The method of claim 42, wherein the adverse event is a bleeding event.

44. The method of claim 43, wherein the bleeding event is gastrointestinal bleeding.

45. The method of claim 42, wherein the adverse event is an adverse cardiovascular event.

46. The method of claim 45, wherein the adverse cardiovascular event is cardiothrombosis.

47. The method of claim 1, wherein the administration of etoricoxib results in greater pain reduction as compared to administration of acetaminophen to the subject.

48. The method of any one of claims 32-47, wherein the acetaminophen is administered as a dose of about 1 g/day, about 2 g/day, about 3 g/day, about 4 g/day, or about 5 g/day.

49. A method for treating a subject infected with a virus capable of causing Viral Hemorrhagic Fever (VHF), the method comprising:
assessing the subject for one or more symptoms of VHF, and
if one or more symptoms of VHF is present, administering to the subject a pharmaceutical composition comprising a therapeutically effective amount of etoricoxib or a pharmaceutically acceptable salt thereof.

50. The method of claim 49, further comprising:
reassessing symptoms following the administration of the pharmaceutical composition; and
adjusting treatment according to the reassessed symptoms, wherein the adjusting comprises administering further the pharmaceutical composition if the reassessed symptoms are worse, not improved or improved but not gone compared to the assessed symptoms and terminating administering the pharmaceutical composition if the reassessed symptoms are gone compared to the assessed symptoms.

51. The method of any one of claims 49-50, wherein the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

52. The method of claim 49, wherein the VHF is caused by one or more RNA viruses derived from a family of viruses selected from Arenaviridae, Bunyaviridae, Filoviridae, Flaviviridae, and any combinations thereof.

53. The method of claim 52, wherein the VHF is caused by one or more RNA viruses from the Flaviviridae family of viruses.

54. The method of claim 53, wherein the VHF is caused by a Dengue Virus.

55. The method of any one of claims 49-50, wherein the therapeutically effective amount of the etoricoxib or a pharmaceutically acceptable salt thereof causes a reduction in viral load, viral titer, or viral shedding.

56. The method of claim 49, wherein the pharmaceutical composition is administered at a duration and a dosage such that the subject experiences a reduction in the one or more symptoms.

57. The method of claim 56, wherein the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

58. The method of any one of claims 49-50, wherein the therapeutically effective amount of the etoricoxib or a pharmaceutically acceptable salt thereof is provided in a quantity sufficient for a single course of therapy.

59. The method of claim 49-50, wherein the pharmaceutical composition is provided in a single course of therapy package.

60. The method of claim 49, wherein the pharmaceutical composition is administered at a dosing schedule matched to the time course of the VHF.

61. The method of claim 60, wherein the pharmaceutical composition comprises a dose of etoricoxib sufficient to achieve a maximum anti-pyretic effect during the febrile phase of the infection.

62. The method of claim 61, further comprising administering a lower maintenance dose during the critical phase and/or recovery phase of the infection compared to the dose administered during the febrile phase of the infection.

63. The method of any one of claims 49-50, wherein the pharmaceutical composition is administered at a dose that does not affect platelet aggregation or bleeding time.

64. The method of any one of claims 49-50, wherein the pharmaceutical composition is administered at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

65. The method of any one of claims 49-50, wherein the pharmaceutical composition is administered as an initial loading dose of about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

66. The method of claim 65, wherein the initial loading dose is administered during the febrile phase of the infection.

67. The method of claim 65, further comprising administering a maintenance dose of about 30 mg/day, about 40 mg/day, about 50 mg/day about 60 mg/day, about 70 mg/day, about 80 mg/day, about 90 mg/day, about 100 mg/day, about 110 mg/day, or about 120 mg/day.

68. The method of claim 67, wherein the maintenance dose is administered for up to 3 days, up to 5 days, up to 7 days, up to 10 days, or up to 14 days.

69. The method of any one of claims 67-68, wherein the maintenance dose is administered during the critical phase and/or recovery phase of the infection.

70. The method of claim 67, wherein the maintenance dose is administered until the subject experiences a reduction in the one or more symptoms or until the one or more symptoms resolve.

71. The method of any one of claims 49-50, wherein the pharmaceutical composition is suitable for oral administration.

72. The method of claim 71, wherein the pharmaceutical composition comprises a solid oral dosage formulation.

73. The method of claim 72, wherein the solid oral dosage formulation is a tablet.

74. The method of claim 72, wherein the solid oral dosage formulation is a capsule.

75. The method of any one of claims 73-74, wherein the solid oral dosage formulation is provided in a blister pack container.

76. The method of claim 71, wherein the pharmaceutical composition comprises an oral suspension formulation.

77. The method of claim 71, wherein the pharmaceutical composition comprises a liquid solution formulation.

78. The method of claim 49, further comprising a second pharmaceutical composition comprising an active agent or a pharmaceutically acceptable salt thereof, wherein the active agent is not etoricoxib.

79. The method of claim 78, wherein the active agent or a pharmaceutically acceptable salt thereof is acetaminophen (paracetamol).

80. The method of any one of claims 49-50, wherein the method does not increase the risk of an adverse event as compared to administration of acetaminophen to the subject.

81. The method of claim 80, wherein the adverse event is a bleeding event.

82. The method of claim 81, wherein the bleeding event is gastrointestinal bleeding.

83. The method of claim 80, wherein the adverse event is an adverse cardiovascular event.

84. The method of claim 83, wherein the adverse cardiovascular event is cardiothrombosis.

85. The method of any one of claims 49-50, wherein the method does not increase the rate of adverse events as compared to administration of acetaminophen to the subject.

86. The method of claim 85, wherein the adverse event is a bleeding event.

87. The method of claim 86, wherein the bleeding event is gastrointestinal bleeding.

88. The method of claim 85, wherein the adverse event is an adverse cardiovascular event.

89. The method of claim 88, wherein the adverse cardiovascular event is cardiothrombosis.

90. The method of any one of claims 49-50, wherein the method results in fewer adverse events as compared to administration of acetaminophen to the subject.

91. The method of claim 90, wherein the adverse event is a bleeding event.

92. The method of claim 91, wherein the bleeding event is gastrointestinal bleeding.

93. The method of claim 90, wherein the adverse event is an adverse cardiovascular event.

94. The method of claim 93, wherein the adverse cardiovascular event is cardiothrombosis.

95. The method of any one of claims 49-50, wherein the administration of etoricoxib results in greater pain reduction as compared to administration of acetaminophen to the subject.

96. The method of any one of claims 80-95, wherein the acetaminophen is administered as a dose of about 1 g/day, about 2 g/day, about 3 g/day, about 4 g/day, or about 5 g/day.

97. A method for treating a subject infected with a virus capable of causing Viral Hemorrhagic Fever (VHF), the method comprising:
administering to the subject a pharmaceutical composition comprising an active agent or a pharmaceutically acceptable salt thereof at a sufficient dose to cause a reduction in viral load, viral titer, or viral shedding of the virus.

98. The method of claim 97, wherein the active agent is etoricoxib and wherein the pharmaceutical composition further comprises a pharmaceutically acceptable carrier.

99. The method of any one of claims 97-98, wherein the VHF is caused by one or more RNA viruses derived from a family of viruses selected from Arenaviridae, Bunyaviridae, Filoviridae, Flaviviridae, and any combinations thereof.

100. The method of claim 99, wherein the VHF is caused by one or more RNA viruses from the Flaviviridae family of viruses.

101. The method of claim 100, wherein the VHF is caused by a Dengue Virus.

102. The method of any one of claims 97-98, wherein the pharmaceutical composition is provided in a quantity sufficient for a single course of therapy.

103. The method of claim 97-98, wherein the pharmaceutical composition is provided in a single course of therapy package.

104. The method of any one of claims 97-98, wherein the pharmaceutical composition is administered at a dosing schedule matched to the time course of the VHF.

105. The method of claim 104, wherein the pharmaceutical composition comprises a dose of etoricoxib sufficient to achieve a maximum anti-pyretic effect during the febrile phase of an infection.

106. The method of claim 105, further comprising administering a lower maintenance dose during the critical phase and/or recovery phase of the infection compared to the dose administered during the febrile phase of the infection.

107. The method of any one of claims 97-98, wherein the pharmaceutical composition is administered at a dose that does not affect platelet aggregation or bleeding time.

108. The method of any one of claims 97-98, wherein the pharmaceutical composition is administered at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

109. The method of any one of claims 97-98, wherein the pharmaceutical composition is administered as an initial loading dose of about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

110. The method of claim 109, the initial loading dose is administered during the febrile phase of the infection.

111. The method of claim 110, further comprising administering a maintenance dose of about 30 mg/day, about 40 mg/day, about 50 mg/day about 60 mg/day, about 70 mg/day, about 80 mg/day, about 90 mg/day, about 100 mg/day, about 110 mg/day, or about 120 mg/day.

112. The method of claim 111, wherein the maintenance dose is administered for up to 3 days, up to 5 days, up to 7 days, up to 10 days, or up to 14 days.

113. The method of any one of claims 111-112, wherein the maintenance dose is administered during the critical phase and/or recovery phase of the infection.

114. The method of any one of claims 97-98, wherein the pharmaceutical composition is suitable for oral administration.

115. The method of claim 114, wherein the pharmaceutical composition comprises a solid oral dosage formulation.

116. The method of claim 115, wherein the solid oral dosage formulation is a tablet.

117. The method of claim 115, wherein the solid oral dosage formulation is a capsule.

118. The method of any one of claims 116-117, wherein the solid oral dosage is provided in a blister pack container.

119. The method of claim 114, wherein the pharmaceutical composition comprises an oral suspension formulation.

120. The method of claim 114, wherein the pharmaceutical composition comprises a liquid solution formulation.

121. The method of any one of claims 97-98, further comprising administering a second pharmaceutical composition comprising an active agent or a pharmaceutically acceptable salt thereof, wherein the active agent is not etoricoxib.

122. The method of claim 121, wherein the active agent is acetaminophen (paracetamol).

123. The method of any one of claims 97-98, wherein the method does not increase the risk of an adverse event as compared to administration of acetaminophen to the subject.

124. The method of claim 123, wherein the adverse event is a bleeding event.

125. The method of claim 124, wherein the bleeding event is gastrointestinal bleeding.

126. The method of claim 123, wherein the adverse event is an adverse cardiovascular event.

127. The method of claim 126, wherein the adverse cardiovascular event is cardiothrombosis.

128. The method of any one of claims 97-98, wherein the method does not increase the rate of adverse events as compared to administration of acetaminophen to the subject.

129. The method of claim 128, wherein the adverse event is a bleeding event.

130. The method of claim 129, wherein the bleeding event is gastrointestinal bleeding.

131. The method of claim 128, wherein the adverse event is an adverse cardiovascular event.

132. The method of claim 131, wherein the adverse cardiovascular event is cardiothrombosis.

133. The method of any one of claims 97-98, wherein the method results in fewer adverse events as compared to administration of acetaminophen to the subject.

134. The method of claim 133, wherein the adverse event is a bleeding event.

135. The method of claim 134, wherein the bleeding event is gastrointestinal bleeding.

136. The method of claim 133, wherein the adverse event is an adverse cardiovascular event.

137. The method of claim 136, wherein the adverse cardiovascular event is cardiothrombosis.

138. The method of any one of claims 97-98, wherein the administration of etoricoxib results in greater pain reduction as compared to administration of acetaminophen to the subject.

139. The method of any one of claims 123-138, wherein the acetaminophen is administered as a dose of about 1 g/day, about 2 g/day, about 3 g/day, about 4 g/day, or about 5 g/day.

140. A method for treating a subject having Dengue fever, the method comprising: administering to the subject a pharmaceutical composition comprising a therapeutically effective amount of etoricoxib or a pharmaceutically acceptable salt thereof.

141. The method of claim 140, wherein the therapeutically effective amount of the etoricoxib or a pharmaceutically acceptable salt thereof causes a reduction in Dengue fever viral load, viral titer, or viral shedding.

142. The method of claim 140, wherein the pharmaceutical composition is administered at a duration and a dosage such that the subject experiences a reduction in one or more Dengue fever symptoms.

143. The method of claim 142, wherein the one or more symptoms is selected from fever, pyrexia, pain, inflammation, myalgia, arthralgia, nausea, vomiting, rash, prostration, headache, photophobia, pharyngitis, cough, diarrhea, constipation, abdominal pain, hyperesthesia, dizziness, confusion, tremor, facial flushing, skin erythema, generalized body ache, and combinations thereof.

144. The method of claim 140, wherein the therapeutically effective amount of the etoricoxib of pharmaceutically acceptable salt thereof is provided in a quantity sufficient for a single course of therapy.

145. The method of claim 140, wherein the therapeutically effective amount of the etoricoxib or a pharmaceutically acceptable salt thereof is provided in a single course of therapy package.

146. The method of claim 140, wherein the therapeutically effective amount of the etoricoxib or a pharmaceutically acceptable salt thereof is administered at a dosing schedule matched to the time course of the Dengue fever.

147. The method of claim 146, wherein the pharmaceutical composition comprises a dose of etoricoxib sufficient to achieve maximum anti-pyretic effect during the febrile phase of the infection.

148. The method of claim 147, further comprising administering a lower maintenance dose during the critical phase and/or recovery phase of the infection compared to the dose administered during the febrile phase of the infection.

149. The method of claim 140, wherein the pharmaceutical composition is administered at a dose that does not affect platelet aggregation or bleeding time.

150. The method of claim 140, wherein the pharmaceutical composition is administered at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

151. The method of claim 140, wherein the pharmaceutical composition is administered as an initial loading dose of about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

152. The method of claim 151, the initial loading dose is administered during the febrile phase of the infection.

153. The method of claim 151, further comprising administering a maintenance dose of about 30 mg/day, about 40 mg/day, about 50 mg/day about 60 mg/day, about 70 mg/day, about 80 mg/day, about 90 mg/day, about 100 mg/day, about 110 mg/day, or about 120 mg/day.

154. The method of claim 153, wherein the maintenance dose is administered for up to 3 days, up to 5 days, up to 7 days, up to 10 days, or up to 14 days.

155. The method of any one of claims 153-154, wherein the maintenance dose is administered during the critical phase and/or recovery phase of the infection.

156. The method of claim 153, wherein the maintenance dose is administered until the subject experiences a reduction in one or more symptoms associated with Dengue fever or until the one or more symptoms resolve.

157. The method of claim 140, wherein the pharmaceutical composition is suitable for oral administration.

158. The method of claim 157, wherein the pharmaceutical composition comprises a solid oral dosage formulation.

159. The method of claim 158, wherein the solid oral dosage formulation is a tablet.

160. The method of claim 158, wherein the solid oral dosage formulation is a capsule.

161. The method of any one of claims 159-160, wherein the solid oral dosage formulation is provided in a blister pack container.

162. The method of claim 157, wherein the pharmaceutical composition comprises an oral suspension formulation.

163. The method of claim 157, wherein the pharmaceutical composition comprises a liquid solution formulation.

164. The method of claim 140, further comprising administering a second pharmaceutical composition comprising an active agent or a pharmaceutically acceptable salt thereof, wherein the active agent is not etoricoxib.

165. The method of claim 164, wherein the active agent is acetaminophen (paracetamol).

166. The method of claim 140, wherein the method does not increase the risk of an adverse event as compared to administration of acetaminophen to the subject.

167. The method of claim 166, wherein the adverse event is a bleeding event.

168. The method of claim 167, wherein the bleeding event is gastrointestinal bleeding.

169. The method of claim 166, wherein the adverse event is an adverse cardiovascular event.

170. The method of claim 169, wherein the adverse cardiovascular event is cardiothrombosis.

171. The method of claim 140, wherein the method does not increase the rate of adverse events as compared to administration of acetaminophen to the subject.

172. The method of claim 171, wherein the adverse event is a bleeding event.

173. The method of claim 172, wherein the bleeding event is gastrointestinal bleeding.

174. The method of claim 171, wherein the adverse event is an adverse cardiovascular event.

175. The method of claim 174, wherein the adverse cardiovascular event is cardiothrombosis.

176. The method of claim 140, wherein the method results in fewer adverse events as compared to administration of acetaminophen to the subject.

177. The method of claim 176, wherein the adverse event is a bleeding event.

178. The method of claim 177, wherein the bleeding event is gastrointestinal bleeding.

179. The method of claim 176, wherein the adverse event is an adverse cardiovascular event.

180. The method of claim 179, wherein the adverse cardiovascular event is cardiothrombosis.

181. The method of claim 140, wherein the administration of etoricoxib results in greater pain reduction as compared to administration of acetaminophen to the subject.

182. The method of any one of claims 166-181, wherein the acetaminophen is administered as a dose of about 1 g/day, about 2 g/day, about 3 g/day, about 4 g/day, or about 5 g/day.

183. A product for short-term use of etoricoxib by a subject in need thereof, the product comprising a single course therapy of etoricoxib or a pharmaceutically acceptable salt thereof.

184. The product of claim 183, further comprising a container having a pre-defined number of solid oral dosage forms of the etoricoxib or a pharmaceutically acceptable salt thereof.

185. The product of claim 183, wherein the solid oral dosage forms further comprise a pharmaceutically acceptable carrier.

186. The product of claim 183, wherein the single course therapy of etoricoxib or a pharmaceutically acceptable salt thereof comprises about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day.

187. The product of claim 183, wherein the single course therapy of etoricoxib or a pharmaceutically acceptable salt thereof comprises an initial loading dose of about 90 mg/day, about 100 mg/day, about 110 mg/day, about 120 mg/day, about 130 mg/day, or about 140 mg/day, about 150 mg/day, about 160 mg/day, about 170 mg/day, about 180 mg/day, about 190 mg/day, about 200 mg/day, about 210 mg/day, about 220 mg/day, about 230 mg/day, about 240 mg/day, about 250 mg/day, about 260 mg/day, about 270 mg/day, about 280 mg/day, about 290 mg/day, about 300 mg/day, about 310 mg/day, about 320 mg/day, about 330 mg/day, about 340 mg/day, about 350 mg/day, about 360 mg/day, about 370 mg/day, about 380 mg/day, about 390 mg/day, about 400 mg/day, about 410 mg/day, about 420 mg/day, about 430 mg/day, about 440 mg/day, about 450 mg/day, about 460 mg/day, about 470 mg/day, about 480 mg/day, about 490 mg/day, or about 500 mg/day, followed by a maintenance dose of etoricoxib of about 30 mg/day, about 40 mg/day, about 50 mg/day about 60 mg/day, about 70 mg/day, about 80 mg/day, about 90 mg/day, about 100 mg/day, about 110 mg/day, or about 120 mg/day.

188. The product of claim 187, wherein the maintenance dose is provided for up to 3 days, up to 5 days, up to 7 days, up to 10 days, or up to 14 days.

189. The product of claim 184, wherein the solid oral dosage form is a tablet.

190. The product of claim 184, wherein the solid oral dosage form is a capsule.

191. The product of any one of claims 189-190, wherein the solid oral dosage form is provided in a blister pack container.

192. The method of any of claim 1, 49, or 140, wherein the therapeutically effective amount of the etoricoxib or a pharmaceutically acceptable salt thereof is 60 mg, 90 mg, or 120 mg.
